(19) United States
(12) Patent Application Publication
(10) Pub. No.: US 2022/0039697 A1
(43) Pub. Date: Feb. 10, 2022

(54) DETECTION DEVICE

(71) Applicant: Japan Display Inc., Tokyo (JP)

(72) Inventors: Toshiyuki HIGANO, Minato-ku (JP); Kazuhiro NISHIYAMA, Minato-ku (JP); Yasushi TOMIOKA, Minato-ku (JP); Genki ASOZU, Minato-ku (JP); Kazuki MATSUNAGA, Minato-ku (JP)

(73) Assignee: Japan Display Inc., Tokyo (JP)

(21) Appl. No.: 17/452,407

(22) Filed: Oct. 27, 2021

Related U.S. Application Data
(63) Continuation of application No. PCT/JP2020/015786, filed on Apr. 8, 2020.

(30) Foreign Application Priority Data
May 8, 2019 (JP) 2019-088581

(51) Int. Cl. A61B 5/1172; G02F 1/1333; G06K 9/20; G06K 9/00; F21V 8/00

(52) U.S. Cl. CPC A61B 5/1172; G02F 1/1333; G02B 6/005; G06K 9/0004; G06K 9/209

(57) ABSTRACT

A detection device includes: an optical sensor including a sensor base member and photoelectric conversion elements that are provided on the sensor base member and configured to output signals corresponding to light emitted to the photoelectric conversion elements; a light-emitting element configured to emit output light toward a direction of an object to be measured; and an optical element including first light-transmitting areas and a non-light-transmitting area and provided between the optical sensor and the object to be measured. In the optical element, the first light-transmitting areas are provided at positions overlapping the respective photoelectric conversion elements so as to penetrate the optical element in a thickness direction of the optical element and are configured to transmit incident light incident on the photoelectric conversion elements, and the non-light-transmitting area is provided between the first light-transmitting areas and has light transmittance lower than light transmittance of the first light-transmitting areas.

### CROSS-REFERENCE TO RELATED APPLICATION

This application claims the benefit of priority from Japanese Patent Application No. 2019-088581 filed on May 8, 2019 and International Patent Application No. PCT/JP2020/015786 filed on Apr. 8, 2020, the entire contents of which are incorporated herein by reference.

### BACKGROUND

### 1. Technical Field

What is disclosed herein relates to a detection device.

### 2. Description of the Related Art

Japanese Translation of PCT International Application Publication Laid-open No. 2017-527045 (JP-A-2017-527045) describes an image acquisition device that includes a display panel, a light source, a light guide plate, a pinhole imaging plate, and an image sensor. In JP-A-2017-527045, the light source is provided at a side end of the light guide plate. Light emitted from the light source travels in the light guide plate, and light reflected by an object to be detected is incident on the image sensor through the optical pinhole imaging plate.

Japanese Translation of PCT International Application Publication Laid-open No. 2018-506806 (JP-A-2018-206806) describes an electronic device that includes an optical image sensor, a pinhole array mask layer, a display layer, a cover layer, and a light source. In JP-A-206806, the light source can direct light toward a finger of a user and guide the light toward the optical image sensor.

Detection devices including an optical sensor are required to detect not only a shape of a fingerprint of an object to be detected such as a finger or a palm, but also various types of biological information on the object to be detected. In this case, the optical sensor may include a plurality of light sources corresponding to the biological information to be detected, and thus, may be difficult to be smaller in size. The light source of JP-A-2017-527045 has an edge-light structure provided at the side end of the light guide plate, and JP-A-2017-527045 does not describe a configuration of providing the light source directly below the display panel. JP-A-2018-206806 does not describe any specific arrangement of the light source.

### SUMMARY

According to an aspect, a detection device includes: an optical sensor including a sensor base member and a plurality of photoelectric conversion elements that are provided on the sensor base member and configured to output signals corresponding to light emitted to the photoelectric conversion elements; a light-emitting element configured to emit output light toward a direction of an object to be measured; and an optical element including a plurality of first light-transmitting areas and a non-light-transmitting area, and provided between the optical sensor and the object to be measured. In the optical element, the first light-transmitting areas are provided at positions overlapping the respective photoelectric conversion elements so as to penetrate the optical element in a thickness direction of the optical element and are configured to transmit incident light incident on the photoelectric conversion elements, and the non-light-transmitting area is provided between the first light-transmitting areas and has light transmittance lower than light transmittance of the first light-transmitting areas.

### BRIEF DESCRIPTION OF THE DRAWINGS

FIG. 1 is a perspective view illustrating a detection device according to a first embodiment;

FIG. 2 is a II-II′ sectional view of FIG. 1;

FIG. 3 is a partial enlarged view illustrating an area A of FIG. 2 in an enlarged manner;

FIG. 4 is a circuit diagram illustrating a pixel array in a display area;

FIG. 5 is a plan view schematically illustrating an optical sensor;

FIG. 6 is a VI-VI′ sectional view of FIG. 5;

FIG. 7 is a circuit diagram illustrating a partial detection area of a photoelectric conversion element;

FIG. 8 is a VIII-VIII′ sectional view of FIG. 5;

FIG. 9 is a sectional view illustrating a light-emitting element of FIG. 8 in an enlarged manner;

FIG. 10 is a plan view illustrating an optical element;

FIG. 11 is a XI-XI′ sectional view of FIG. 10;

FIG. 12 is a sectional view illustrating an optical element according to a first modification;

FIG. 13 is an explanatory diagram for explaining an arrangement relation between a display panel, the optical element, and the optical sensor in a plan view;

FIG. 14 is a XIV-XIV′ sectional view of FIG. 13;

FIG. 15 is a sectional view illustrating an example of a scattering structure;

FIG. 16 is a sectional view illustrating another example of the scattering structure;

FIG. 17 is a sectional view illustrating a schematic sectional configuration of a detection device according to a second embodiment;

FIG. 18 is a perspective view schematically illustrating a lighting device included in the detection device according to the second embodiment;

FIG. 19 is a plan view illustrating an optical element according to the second embodiment;

FIG. 20 is a sectional view schematically illustrating an arrangement relation between the display panel, the optical element, and the optical sensor according to the second embodiment;

FIG. 21 is a sectional view schematically illustrating an arrangement relation between the display panel, an optical element, and the optical sensor according to a second modification of the second embodiment;

FIG. 22 is a plan view illustrating the optical element according to the second modification;

FIG. 23 is a sectional view illustrating a schematic sectional configuration of a detection device according to a third embodiment;

FIG. 24 is a sectional view schematically illustrating an arrangement relation between the display panel, the optical element, and the optical sensor according to the third embodiment;

FIG. 25 is a sectional view schematically illustrating an arrangement relation between the display panel, the optical element, and the optical sensor according to a third modification of the third embodiment;

FIG. 26 is a sectional view illustrating a schematic sectional configuration of a detection device according to a fourth embodiment;

FIG. 27 is a perspective view schematically illustrating a display panel included in the detection device according to the fourth embodiment;

FIG. 28 is a circuit diagram illustrating a drive circuit for the display panel according to the fourth embodiment;

FIG. 29 is an explanatory diagram for explaining an arrangement relation between the display panel, optical elements, and the optical sensor according to the fourth embodiment in the plan view;

FIG. 30 is an explanatory diagram for explaining an arrangement in a pixel according to a fourth modification of the fourth embodiment; and

FIG. 31 is a sectional view illustrating a schematic sectional configuration of a detection device according to a fifth modification of the fourth embodiment.

### DETAILED DESCRIPTION

The following describes aspects (embodiments) for carrying out the present disclosure in detail with reference to the drawings. The present disclosure is not limited to the description of the embodiments given below. Components described below include those easily conceivable by those skilled in the art or those substantially the same. Moreover, the components described below can be combined as appropriate. What is disclosed herein is merely an example, and appropriate modifications maintaining the gist of the disclosure and easily conceivable by those skilled in the art naturally fall within the scope of the present disclosure. To further clarify the description, the drawings schematically illustrate, for example, widths, thicknesses, and shapes of various parts as compared with actual aspects thereof, in some cases. However, they are merely examples, and interpretation of the present disclosure is not limited thereto. The same element as that illustrated in a drawing that has already been discussed is denoted by the same reference numeral throughout the description and the drawings, and detailed description thereof will not be repeated in some cases where appropriate.

In this disclosure, when an element is described as being “on/upon” another element, the element can be directly on the other element, or there can be one or more elements between the element and the other element.

### First Embodiment

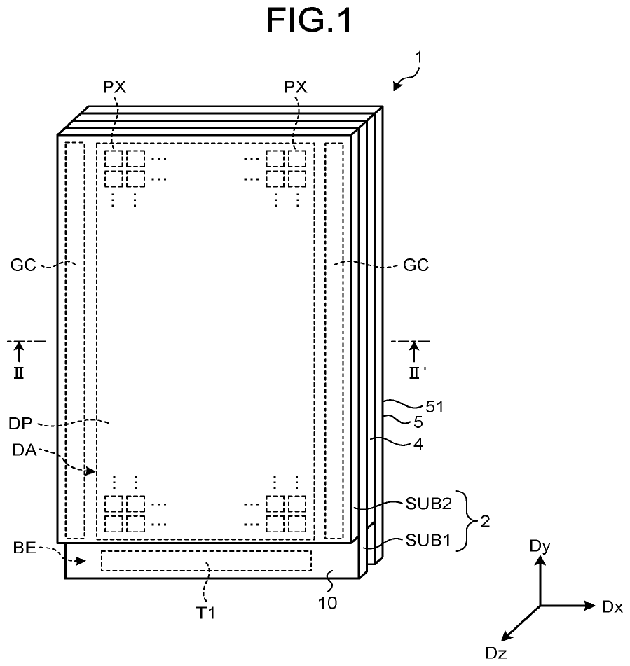

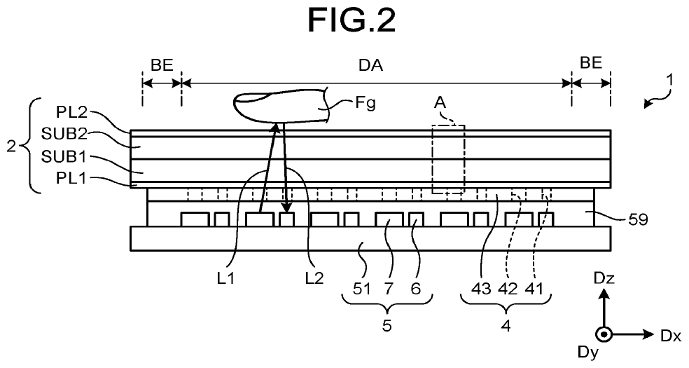

FIG. 1 is a perspective view illustrating a detection device according to a first embodiment. FIG. 2 is a II-II′ sectional view of FIG. 1. As illustrated in FIG. 1, a detection device 1 includes a display panel 2, an optical element 4, and an optical sensor 5. The optical sensor 5, the optical element 4, and the display panel 2 are stacked in a third direction Dz in the order as listed.

A first direction Dx and a second direction Dy are directions parallel to a surface of a sensor base member 51 serving as a base body of the optical sensor 5. The first direction Dx is orthogonal to the second direction Dy. The first direction Dx may, however, intersect the second direction Dy without being orthogonal thereto. The third direction Dz is a direction orthogonal to the first direction Dx and the second direction Dy. The third direction Dz corresponds to, for example, a normal direction to the sensor base member 51. Hereinafter, the term “plan view” refers to a positional relation as viewed from the third direction Dz.

The display panel 2 has a display area DA and a peripheral area BE. The display area DA is an area that is disposed so as to overlap a display part DP and displays an image. The peripheral area BE is an area not overlapping the display part DP and is disposed outside the display area DA.

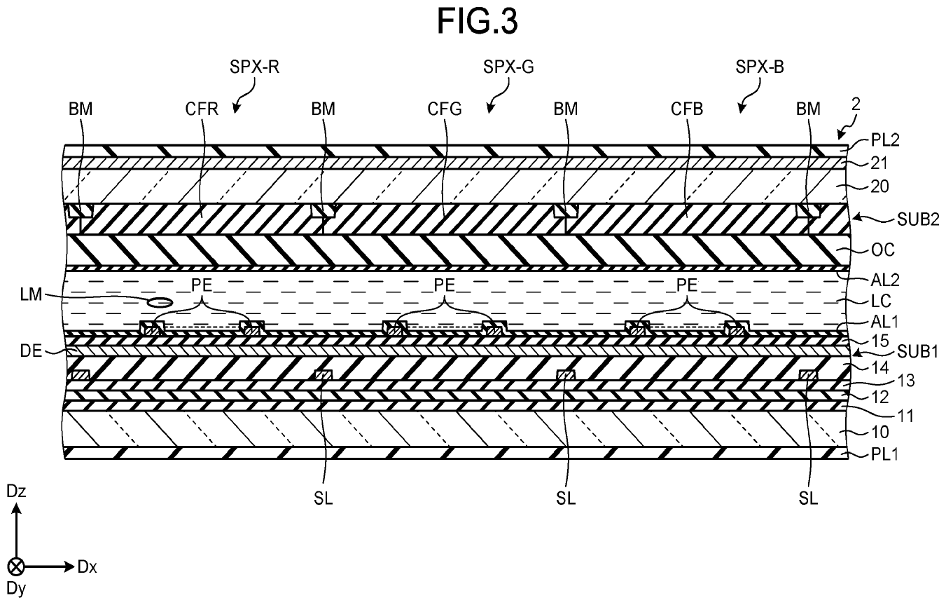

The display panel 2 is a liquid crystal display panel including a liquid crystal layer LC (refer to FIG. 3) as a display element. The display panel 2 includes an array substrate SUB1 and a counter substrate SUB2. The array substrate SUB1 includes a first substrate 10, pixels PX, peripheral circuits GC, and coupling terminals T1. The first substrate 10, a plurality of transistors, a plurality of capacitors, and various types of wiring constitute the array substrate SUB1 for driving each of the pixels PX. The array substrate SUB1 is a drive circuit substrate and is also called a “backplane” or an “active matrix substrate”. A drive integrated circuit (IC) is coupled through the coupling terminals T1.

The display part DP includes the pixels PX, and the pixels PX are arranged in the first direction Dx and the second direction Dy in the display area DA. The peripheral circuits GC and the coupling terminals T1 are provided in the peripheral area BE. The peripheral circuits GC are circuits that drive a plurality of scan lines GL based on various control signals from the drive IC. The peripheral circuits GC sequentially or simultaneously select the scan lines GL and supply gate drive signals to the selected scan lines GL. Through this operation, the peripheral circuits GC select the pixels PX coupled to the scan lines GL.

The drive IC is a circuit that controls display of the display panel 2. The drive IC may be mounted as a chip on film (COF) on a flexible printed circuit board or a rigid substrate coupled to the coupling terminals T1. The drive IC is not limited thereto and may be mounted as a chip on glass (COG) in the peripheral area BE of the first substrate 10.

As illustrated in FIG. 2, the display panel 2 is provided between a light-emitting element 7 of the optical sensor 5 and a finger Fg serving as an object to be measured. The optical sensor 5 includes the sensor base member 51, a plurality of photoelectric conversion elements 6, and the light-emitting elements 7. The sensor base member 51 is an insulting base member, and is, for example, a glass substrate. The sensor base member 51 may, alternatively, be a resin substrate or a resin film formed of a resin such as polyimide. The photoelectric conversion elements 6 and the light-emitting elements 7 are provided on the same sensor base member 51. The photoelectric conversion elements 6 and the light-emitting elements 7 are provided in an area of the sensor base member 51 overlapping the display area DA. The photoelectric conversion elements 6 and the light-emitting elements 7 may, however, be provided in an area partially overlapping the display area DA.

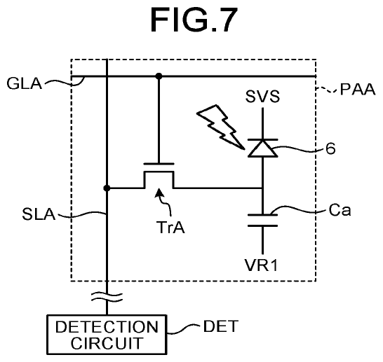

Each of the photoelectric conversion elements 6 is, for example, a photodiode formed of, for example, amorphous silicon. The photoelectric conversion element 6 outputs, to a detection circuit DET (refer to FIG. 7), an electrical signal corresponding to light L2 to be emitted.

For example, an inorganic light-emitting element (light-emitting diode (LED)) or an organic electroluminescent (EL) diode (organic light-emitting diode (OLED)) is used as the light-emitting element 7. The display panel 2 is provided so as to face the sensor base member 51 with the optical element 4 interposed therebetween. The light-emitting element 7 emits light L1 toward the display panel 2 and the finger Fg serving as the object to be measured.

The optical element 4 is provided between the optical sensor 5 and the display panel 2 in the third direction Dz. The optical element 4 has a flat plate shape and is provided in an area overlapping at least the photoelectric conversion elements 6 and the light-emitting elements 7. The optical element 4 includes first light-transmitting areas 41, second light-transmitting areas 42, and a non-light-transmitting area 43. The first light-transmitting areas 41 are provided at positions overlapping the respective photoelectric conversion elements 6 so as to penetrate the optical element 4 in a thickness direction of the optical element 4. Each of the first light-transmitting areas 41 has translucency and transmits the light L2 (incident light) incident on the photoelectric conversion element 6.

The second light-transmitting areas 42 are provided at positions overlapping the respective light-emitting elements 7 so as to penetrate the optical element 4 in the thickness direction of the optical element 4. The second light-transmitting areas 42 transmit the light L1 (output light) emitted from the light-emitting elements 7. The non-light-transmitting area 43 is provided between the first light-transmitting areas 41 and the second light-transmitting areas 42 and have lower light transmittance than that of the first light-transmitting areas 41 and the second light-transmitting areas 42. That is, the light L1 and the light L2 do not pass through the non-light-transmitting area 43.

With the above-described configuration, the light L1 emitted from the light-emitting elements 7 passes through the second light-transmitting areas 42 to be incident on the display panel 2. The light L1 passes through the display panel 2 and is reflected on a surface of or in the finger Fg. The light L2 reflected by the finger Fg passes through the display panel 2 and the first light-transmitting areas 41 to be incident on the photoelectric conversion elements 6. As a result, the optical sensor 5 can detect information on a living body such as a fingerprint and/or a blood vessel image (vein pattern) of the finger Fg based on the light L2. At the time of display, the display panel 2 can display a display image using the light L1 that has passed through the display panel 2. In this manner, the light-emitting elements 7 serve as both a light source for detection and a light source for display.

The following describes detailed configurations of the display panel 2, the optical element 4, and the optical sensor 5. FIG. 3 is a partial enlarged view illustrating an area A of FIG. 2 in an enlarged manner. FIG. 3 is a sectional view illustrating a schematic sectional structure of the display panel 2 included in the detection device 1. As illustrated in FIG. 3, the counter substrate SUB2 is disposed so as to face a surface of the array substrate SUB1 in a direction orthogonal to the surface. The liquid crystal layer LC is provided between the array substrate SUB1 and the counter substrate SUB2. The array substrate SUB1 includes the first substrate 10 as a base body. The counter substrate SUB2 includes a second substrate 20 as a base body. The first substrate 10 and the second substrate 20 are formed of, for example, a light-transmitting material such as a glass substrate or a resin substrate.

The array substrate SUB1 includes a first insulating film 11, a second insulating film 12, a third insulating film 13, a fourth insulating film 14, a fifth insulating film 15, pixel signal lines SL, pixel electrodes PE, a common electrode DE, and a first orientation film AL1, on a side of the first substrate 10 facing the counter substrate SUB2.

Herein, in the specification, in a direction orthogonal to the first substrate 10, a direction from the first substrate 10 toward the second substrate 20 will be referred to as “upper side” or simply as “upon”, and a direction from the second substrate 20 toward the first substrate 10 will be referred to as “lower side” or simply as “below”.

The first insulating film 11 is provided upon the first substrate 10. The second insulating film 12 is provided upon the first insulating film 11. The third insulating film 13 is provided upon the second insulating film 12. The signal lines SL are provided upon the third insulating film 13. The fourth insulating film 14 is provided upon the third insulating film 13 and covers the pixel signal lines SL. Although not illustrated in FIG. 3, the scan lines GL are provided, for example, upon the second insulating film 12.

The common electrode DE is provided upon the fourth insulating film 14. The common electrode DE is continuously provided over the display area DA. The common electrode DE is, however, not limited to this configuration, and may be provided with slits and divided into a plurality of pieces. The common electrode DE is covered with the fifth insulating film 15.

The pixel electrodes PE are provided upon the fifth insulating film 15 and face the common electrode DE with the fifth insulating film 15 interposed therebetween. The pixel electrodes PE and the common electrode DE are formed of, for example, a light-transmitting conductive material such as indium tin oxide (ITO) or indium zinc oxide (IZO). The pixel electrodes PE and the fifth insulating film 15 are covered with the first orientation film AL1.

The first insulating film 11, the second insulating film 12, the third insulating film 13, and the fifth insulating film 15 are formed of, for example, a light-transmitting inorganic material such as a silicon oxide or a silicon nitride. The fourth insulating film 14 is formed of a light-transmitting resin material and has a film thickness greater than those of the other insulating films formed of the inorganic material.

The counter substrate SUB2 includes, for example, a light-blocking layer BM, color filters CFR, CFG, and CFB, an overcoat layer OC, and a second orientation film AL2, on a side of the second substrate 20 facing the array substrate SUB1. The counter substrate SUB2 includes a conductive layer 21 on a side of the second substrate 20 opposite to the array substrate SUB1.

In the display area DA, the light-blocking layer BM is located on the side of the second substrate 20 facing the array substrate SUB1. The light-blocking layer BM defines openings facing the respective pixel electrodes PE. Each of the pixel electrodes PE is partitioned off for each of the openings of the pixels PX. The light-blocking layer BM is formed of a black resin material or a light-blocking metal material.

Each of the color filters CFR, CFG, and CFB is located on the side of the second substrate 20 facing the array substrate SUB1 and overlaps, at ends thereof, the light-blocking layer BM. In an example, the color filters CFR, CFG, and CFB are respectively formed of resin materials colored in red, green, and blue.

The overcoat layer OC covers the color filters CFR, CFG, and CFB. The overcoat layer OC is formed of a light-transmitting resin material. The second orientation film AL2 covers the overcoat layer OC. Each of the first orientation film AL1 and the second orientation film AL2 is formed of, for example, a material that exhibits a horizontal orientation property.

The conductive layer 21 is provided upon the second substrate 20. The conductive layer 21 is, for example, of a light-transmitting conductive material such as ITO. Externally applied static electricity and static electricity charging a second polarizing plate PL2 flow through the conductive layer 21. The detection device 1 can remove the static electricity in a short time, and thus, can reduce the static electricity applied to the liquid crystal layer LC that serves as a display layer. As a result, the display panel 2 can be improved in electrostatic discharge (ESD) resistance.

A first polarizing plate PL1 is disposed on an external surface of the first substrate 10, or a surface thereof facing the optical element 4 (refer to FIG. 2). The second polarizing plate PL2 is disposed on an external surface of the second substrate 20, or a surface on an observation position side thereof. A first polarization axis of the first polarizing plate PL1 and a second polarization axis of the second polarizing plate PL2 are in a positional relation of, for example, crossed Nicols in an XY-plane. The display panel 2 may include other optical functional elements, such as a retardation film, in addition to the first polarizing plate PL1 and the second polarizing plate PL2.

The array substrate SUB1 and the counter substrate SUB2 are disposed such that the first orientation film AL1 and the second orientation film AL2 face each other. The liquid crystal layer LC is sealed between the first orientation film AL1 and the second orientation film AL2. The liquid crystal layer LC is formed of a negative liquid crystal material having a negative dielectric anisotropy or a positive liquid crystal material having a positive dielectric anisotropy.

For example, in the case where the liquid crystal layer LC is a negative liquid crystal material, when no voltage is applied to the liquid crystal layer LC, a long axis of a liquid crystal molecule LM is initially oriented in a direction along the first direction Dx in an XY-plane. In contrast, in a state where a voltage is applied to the liquid crystal layer LC, that is, in an on-state where an electric field is formed between the pixel electrodes PE and the common electrode DE, the orientation state of the liquid crystal molecule LM changes under the influence of the electric field. In the on-state, the polarization state of linearly polarized incident light changes in accordance with the orientation state of the liquid crystal molecule LM when the light passes through the liquid crystal layer LC.

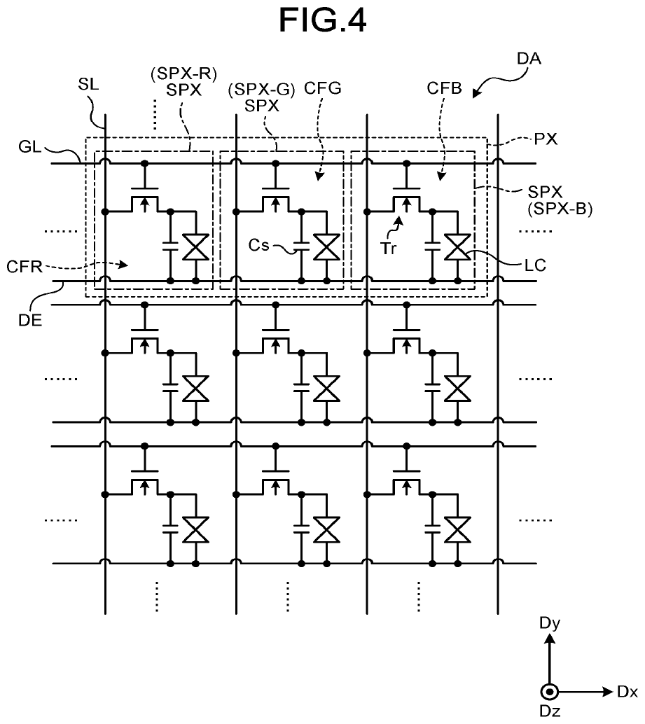

FIG. 4 is a circuit diagram illustrating a pixel array in the display area. For example, the array substrate SUB1 is provided with switching elements Tr of respective sub-pixels SPX, the pixel signal lines SL, and the scan lines GL illustrated in FIG. 4. The pixel signal lines SL extend in the second direction Dy. The pixel signal lines SL are wiring for supplying pixel signals to the respective pixel electrodes PE (refer to FIG. 3). The scan lines GL extend in the first direction Dx. The scan lines GL are wiring for supplying drive signals (scan signals) for driving the respective switching elements Tr.

Each of the pixels PX includes the sub-pixels SPX. Each of the sub-pixels SPX includes a corresponding one of the switching elements Tr and a capacitance of the liquid crystal layer LC. The switching element Tr includes a thin-film transistor, and in this example, an n-channel metal-oxide-semiconductor (MOS) thin-film transistor (TFT). The fifth insulating film 15 is provided between the pixel electrodes PE and the common electrode DE illustrated in FIG. 3, and these components provide a storage capacitor Cs illustrated in FIG. 4.

Color regions colored in three colors of, for example, red (R), green (G), and blue (B) are periodically arranged as the color filters CFR, CFG, and CFB illustrated in FIG. 3. The color regions of the three colors of R, G, and B are associated with sub-pixels SPX-R, SPX-G, and SPX-B as one set. The sub-pixels SPX corresponding to the color regions of the three colors constitute the pixel PX as one set. That is, the display panel 2 includes the sub-pixel SPX-R for displaying a red color, the sub-pixel SPX-G for displaying a green color, and the sub-pixel SPX-B for displaying a blue color. The color filters may include color regions of four or more colors. In this case, each pixel PX may include four or more of the sub-pixels SPX.

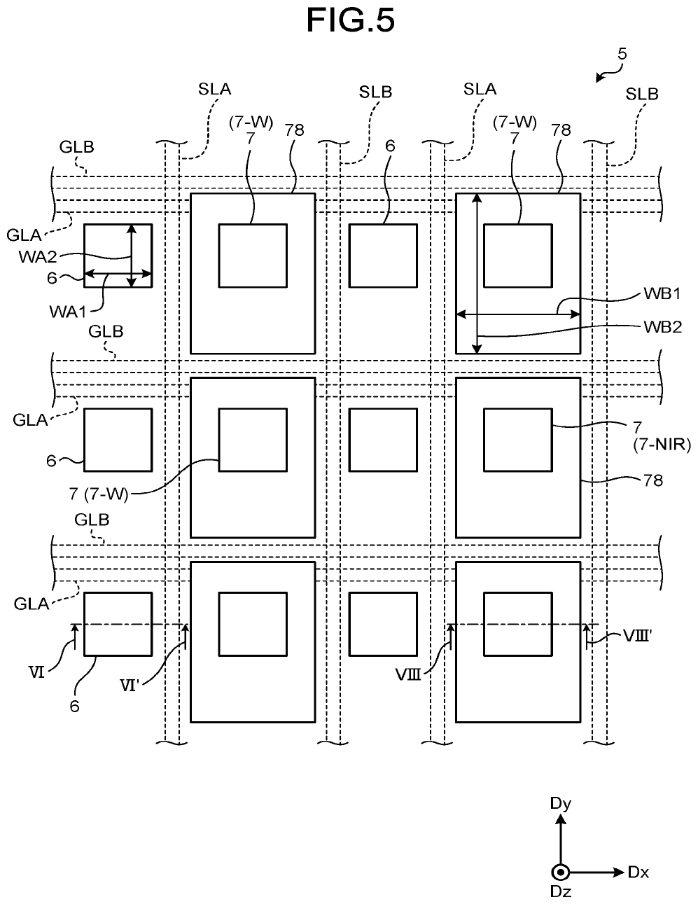

FIG. 5 is a plan view schematically illustrating the optical sensor. As illustrated in FIG. 5, the photoelectric conversion elements 6 and the light-emitting elements 7 are arranged in the first direction Dx and the second direction Dy. Specifically, the photoelectric conversion elements 6 and the light-emitting elements 7 are alternately arranged in the first direction Dx. The photoelectric conversion elements 6 and the light-emitting elements 7 are arranged in the second direction Dy. The light-emitting elements 7 are arranged adjacent to the photoelectric conversion elements 6. The light-emitting elements 7 are arranged in a one-to-one relation with the photoelectric conversion elements 6. However, the light-emitting elements 7 are not limited to this arrangement and may be provided one for the multiple photoelectric conversion elements 6. In this case, the photoelectric conversion elements 6 include the photoelectric conversion elements 6 adjacent to the light-emitting elements 7 in the first direction Dx and the photoelectric conversion elements 6 adjacent to the other photoelectric conversion elements 6 in the first direction Dx without the light-emitting elements 7 interposed therebetween.

The sensor base member 51 is provided with various types of wiring including, for example, sensor signal lines SLA, sensor scan lines GLA, light source signal lines SLB, and light source scan lines GLB. The sensor scan lines GLA are wiring for supplying drive signals (scan signals) for driving sensor switching elements TrA (refer to FIG. 7). With this configuration, the photoelectric conversion elements 6 are sequentially selected. The sensor signal lines SLA are wiring for outputting detection signals of the photoelectric conversion elements 6 to the detection circuit DET (refer to FIG. 7).

The light source scan lines GLB are wiring for supplying drive signals (scan signals) for driving switching elements included in drive circuits for the light-emitting elements 7. The light source signal lines SLB are wiring for supplying drive voltages to the light-emitting elements 7.

Each of the sensor scan lines GLA and the light source scan lines GLB extends in the first direction Dx. Each of the sensor signal lines SLA and the light source signal lines SLB extends in the second direction Dy. The photoelectric conversion elements 6 are provided in areas surrounded by the sensor scan lines GLA and the sensor signal lines SLA. The light-emitting elements 7 are provided in areas surrounded by the light source scan lines GLB and the light source signal lines SLB. Drive circuits for driving the photoelectric conversion elements 6 and the light-emitting elements 7 are provided in respective areas surrounded by the sensor scan lines GLA, the light source scan lines GLB, the sensor signal lines SLA, and the light source signal lines SLB.

An anode electrode 78 is coupled to the light-emitting element 7. The anode electrode 78 has a larger area than that of the light-emitting element 7 in the plan view. The anode electrode 78 is formed of a metal material such as silver (Ag) and reflects light emitted from a lateral side of the light-emitting element 7 to emit the light L1 toward the display panel 2. That is, an area including the light-emitting element 7 and the anode electrode 78 serves as a light-emitting surface for emitting the light L1.

A width WB1 in the first direction Dx of the anode electrode 78 is greater than a width WA1 in the first direction Dx of the photoelectric conversion element 6. A width WB2 in the second direction Dy of the anode electrode 78 is greater than a width WA2 in the second direction Dy of the photoelectric conversion element 6. With this configuration, the light-emitting elements 7 can well emit the light L1 to the entire display area DA.

The light-emitting elements 7 include first light-emitting elements 7-W and second light-emitting elements 7-NIR, and the first light-emitting element 7-W and the second light-emitting element 7-NIR emit the light L1 having different wavelengths. The first light-emitting elements 7-W emit visible light (for example, white light). The first light-emitting elements 7-W may be composed of a plurality of light-emitting elements or may be composed of combinations each of which includes one or more light-emitting elements and one or more fluorescent bodies. The second light-emitting elements 7-NIR emit, for example, near-infrared light. One second light-emitting element 7-NIR is provided for the multiple first light-emitting elements 7-W (three of the first light-emitting elements 7-W in the example illustrated in FIG. 5).

With this configuration, when the display panel 2 performs display, the first light-emitting elements 7-W among the light-emitting elements 7 emit the light L1, and when the optical sensor 5 performs detection, the first light-emitting elements 7-W and the second light-emitting elements 7-NIR among the light-emitting elements 7 emit the light L1. The light-emitting elements 7 are not limited to light-emitting elements emitting the white and near-infrared light L1 and may include light-emitting elements that emit the light L1 having another wavelength. The light L1 having different wavelengths may be emitted depending on the information on the living body to be detected by the optical sensor 5, such as asperities (fingerprint), the blood vessel image, a pulse wave, pulsation, or a blood oxygen concentration of the finger Fg or a palm. For example, in the case of the fingerprint detection, the optical sensor 5 may perform the detection based on the visible light emitted from the first light-emitting elements 7-W, and in the case of the detection of the blood vessel image (vein pattern), the optical sensor 5 may perform the detection based on the near-infrared light emitted from the second light-emitting elements 7-NIR.

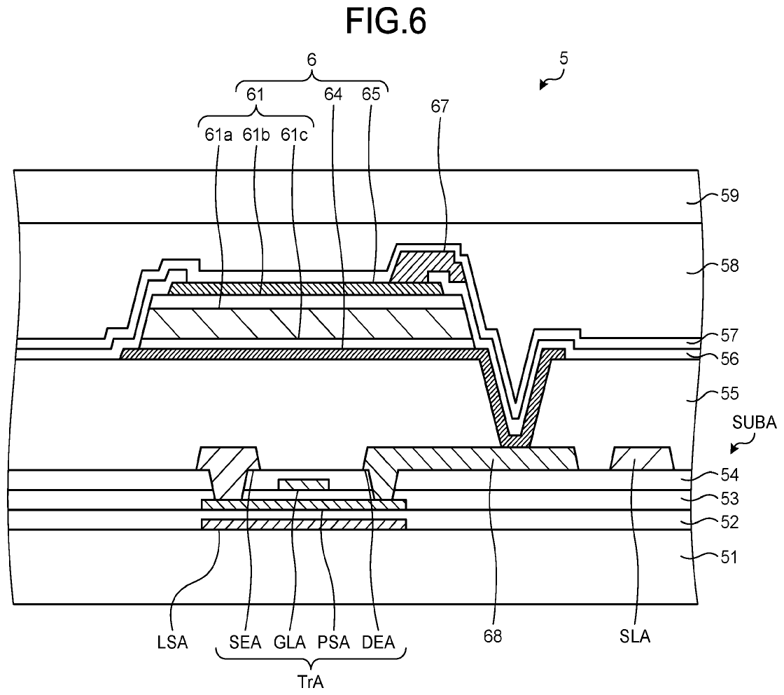

FIG. 6 is a VI-VI′ sectional view of FIG. 5. FIG. 6 schematically illustrates a sectional configuration of the photoelectric conversion element 6 and one of the sensor switching elements TrA. The sensor switching elements TrA are provided so as to correspond to the photoelectric conversion elements 6. Each of the sensor switching elements TrA includes a thin-film transistor, and in this example, includes an n-channel MOSTFT.

As illustrated in FIG. 6, a lower electrode 64, a semiconductor 61, and an upper electrode 65 of the photoelectric conversion element 6 are stacked upon a first organic insulating layer 55 of a sensor array substrate SUBA in the order of the lower electrode 64, the semiconductor 61, and the upper electrode 65. That is, the lower electrode 64 faces the upper electrode 65 with the semiconductor 61 serving as a photoelectric conversion layer interposed therebetween in a direction orthogonal the surface of the sensor base member 51. The sensor array substrate SUBA is a drive circuit substrate that drives the sensor on a predetermined detection area basis. The sensor array substrate SUBA includes, for example, the sensor base member 51, the sensor switching elements TrA, and various types of wiring. The sensor array substrate SUBA also includes various switching elements and various types of wiring for driving the light-emitting elements 7 (refer to FIG. 5).

The photoelectric conversion element 6 is a positive-intrinsic-negative (PIN) photodiode. The semiconductor 61 is of amorphous silicon (a-Si). The semiconductor 61 includes an i-type semiconductor 61a, an n-type semiconductor 61b, and a p-type semiconductor 61c. The i-type semiconductor 61a, the n-type semiconductor 61b, and the p-type semiconductor 61c constitute a specific example of the photoelectric conversion element. In FIG. 6, the p-type semiconductor 61c, the i-type semiconductor 61a, and the n-type semiconductor 61b are stacked in a direction orthogonal to the surface of the sensor base member 51, in the order as listed. However, a reversed configuration may be employed. That is, the semiconductors may be stacked in the order of the n-type semiconductor 61b, the i-type semiconductor 61a, and the p-type semiconductor 61c.

The lower electrode 64 is the anode of the photoelectric conversion element 6 and is an electrode for reading each of the detection signals. The upper electrode 65 is the cathode of the photoelectric conversion element 6 and is an electrode for supplying a power supply signal SVS to the photoelectric conversion element 6.

An insulating layer 56 and an insulating layer 57 are provided upon the first organic insulating layer 55. The insulating layer 56 covers a peripheral portion of the upper electrode 65 and is provided with an opening at a position overlapping the upper electrode 65. Coupling wiring 67 is coupled to the upper electrode 65 at a portion of the upper electrode 65 not provided with the insulating layer 56. The coupling wiring 67 is wiring for coupling the upper electrode 65 to a power supply signal line Lvs. The insulating layer 57 is provided upon the insulating layer 56 so as to cover the upper electrode 65 and the coupling wiring 67. A second organic insulating layer 58 serving as a planarizing layer and an overcoat layer 59 are provided upon the insulating layer 57.

As illustrated in FIG. 6, the sensor switching element TrA is provided on the sensor base member 51. Specifically, a light-blocking layer LSA, an insulating layer 52, a semiconductor layer PSA, an insulating layer 53, each of the sensor scan lines GLA, an insulating layer 54, a source electrode SEA and an anode coupling line 68 (drain electrode DEA), and the first organic insulating layer 55 are provided on one surface of the sensor base member 51 in the order as listed. For example, a silicon oxide (SiO) film, a silicon nitride (SiN) film, or a silicon oxynitride (SiON) film is used as inorganic insulating layers such as the insulating layers 52, 53, 54, 56, and 57. Each of the inorganic insulating layers is not limited to a single layer, but may be a multilayered film.

The light-blocking layer LSA is formed of a material having lower light transmittance than that of the sensor base member 51 and is provided below the semiconductor layer PSA. The insulating layer 52 is provided upon the sensor base member 51 so as to cover the light-blocking layer LSA. The semiconductor layer PSA is provided upon the insulating layer 52. For example, polysilicon or an oxide semiconductor is used as the semiconductor layer PSA.

The insulating layer 53 is provided upon the insulating layer 52 so as to cover the semiconductor layer PSA. The sensor scan line GLA is provided upon the insulating layer 53. A portion of the sensor scan line GLA overlapping the semiconductor layer PSA serves as a gate electrode. The sensor switching element TrA has a top-gate structure in which the sensor scan line GLA is provided on the upper side of the semiconductor layer PSA. However, the sensor switching element TrA is not limited thereto and may have a bottom-gate structure or a dual-gate structure.

The insulating layer 54 is provided upon the insulating layer 53 so as to cover the sensor scan line GLA. The source electrode SEA (signal line SLA) and the drain electrode DEA (anode coupling line 68) are provided upon the insulating layer 54. The source electrode SEA and the drain electrode DEA are each coupled to the semiconductor layer PSA through a contact hole provided in the insulating layers 53 and 54. The lower electrode 64 of the photoelectric conversion element 6 is coupled to the anode coupling line 68 through a contact hole provided in the first organic insulating layer 55.

Although an amorphous silicon material is used as the photoelectric conversion element 6, an organic material, for example, may be used instead. Polysilicon may be used to form a PIN photodiode as the photoelectric conversion element 6.

FIG. 7 is a circuit diagram illustrating a partial detection area of the photoelectric conversion element. A partial detection area PAA is an area surrounded by the sensor signal lines SLA and the sensor scan lines GLA. As illustrated in FIG. 7, the partial detection area PAA includes the photoelectric conversion element 6, a capacitive element Ca, and the sensor switching element TrA. The gate of the sensor switching element TrA is coupled to the sensor scan line GLA. The source of the sensor switching element TrA is coupled to the sensor signal line SLA. The drain of the sensor switching element TrA is coupled to the anode (lower electrode 64) of the photoelectric conversion element 6 and the capacitive element Ca.

The cathode of the photoelectric conversion element 6 is supplied with the power supply signal SVS from a power supply circuit. The capacitive element Ca is also supplied with a reference signal VR1 serving as an initial potential of the capacitive element Ca from the power supply circuit.

When the partial detection area PAA is irradiated with the light L2, a current corresponding to an amount of the light flows through the photoelectric conversion element 6. As a result, an electrical charge is stored in the capacitive element Ca. After the sensor switching element TrA is turned on, a current corresponding to the electrical charge stored in the capacitive element Ca flows through the sensor signal line SLA. The sensor signal line SLA is coupled to the detection circuit DET. As a result, the optical sensor 5 can detect a signal corresponding to the amount of the light emitted to the photoelectric conversion element 6 for each of the partial detection areas PAA. The optical sensor 5 may include a switching circuit for switching between coupling and decoupling of the sensor signal line SLA to and from the detection circuit DET for each of the sensor signal lines SLA.

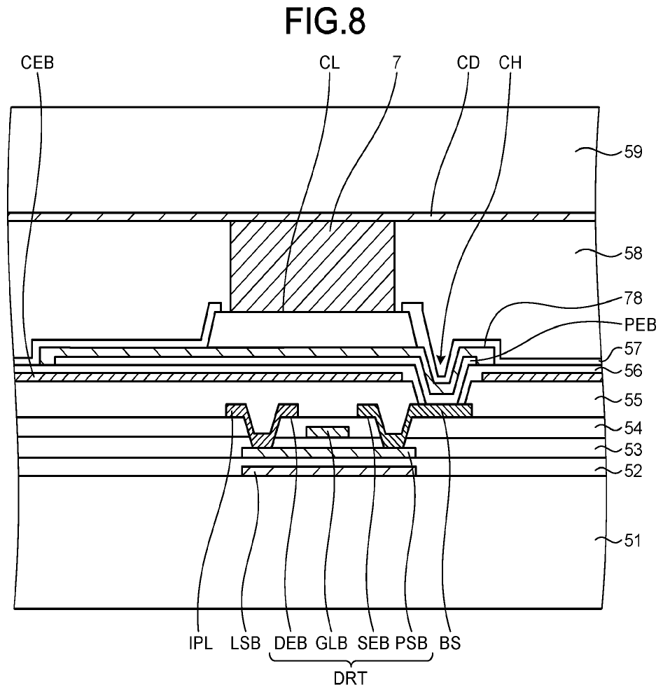

FIG. 8 is a VIII-VIII′ sectional view of FIG. 5. FIG. 8 schematically illustrates a sectional configuration of the light-emitting element 7 and a drive transistor DRT. As illustrated in FIG. 8, the light-emitting element 7 and the drive transistor DRT are provided upon the sensor base member 51.

The drive transistor DRT includes a semiconductor layer PSB, the light source scan line GLB, a drain electrode DEB, and a source electrode SEB. An anode power supply line IPL and a base BS are provided upon the insulating layer 54. A portion of the anode power supply line IPL overlapping the semiconductor layer PSB serves as the drain electrode DEB of the drive transistor DRT. A portion of the base BS overlapping the semiconductor layer PSB serves as the source electrode SEB of the drive transistor DRT. A light-blocking layer LSB is provided below the semiconductor layer PSB. The configuration of the drive transistor DRT is similar to the configuration of the sensor switching element TrA illustrated in FIG. 6, and therefore, detailed description will be omitted.

The first organic insulating layer 55 is provided upon the insulating layer 54 so as to cover the anode power supply line IPL and the base BS. A light source common electrode CEB, an overlapping electrode PEB, and a cathode electrode CD are of indium tin oxide (ITO). The insulating layer 56 is provided between the light source common electrode CEB and the overlapping electrode PEB in the normal direction to the sensor base member 51.

The anode electrode 78 is a layered body of, for example, ITO, silver (Ag), and ITO. The anode electrode 78 is provided upon the overlapping electrode PEB and is coupled to the base BS through a contact hole CH provided in the first organic insulating layer 55. A coupling layer CL is formed of silver paste and is provided upon the anode electrode 78 between the sensor base member 51 and the light-emitting element 7. The light-emitting element 7 is provided upon the coupling layer CL and is electrically coupled to the coupling layer CL. That is, the light-emitting element 7 is electrically coupled to the anode electrode 78 through the coupling layer CL.

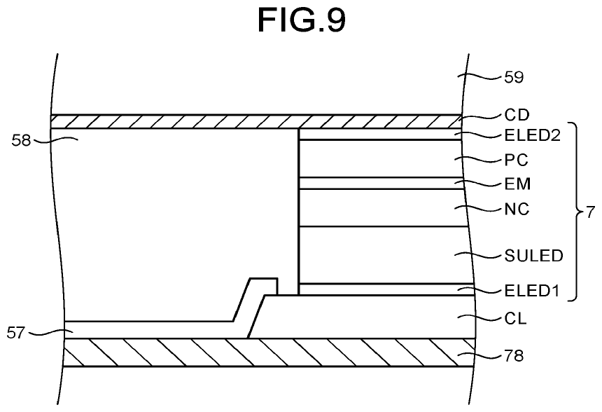

The insulating layer 57 is provided on the insulating layer 56 so as to cover the anode electrode 78 and side surfaces of the coupling layer CL. The second organic insulating layer 58 is provided on the insulating layer 57 so as to cover side surfaces of the light-emitting element 7. The cathode electrode CD is provided on the second organic insulating layer 58 and the light-emitting element 7 and is electrically coupled to a cathode terminal ELED2 of the light-emitting element 7 (refer to FIG. 9). The cathode electrode CD is electrically coupled to the cathode terminals ELED2 of the light-emitting elements 7. The overcoat layer 59 is provided on the cathode electrode CD.

FIG. 9 is a sectional view illustrating the light-emitting element of FIG. 8 in an enlarged manner. As illustrated in FIG. 9, the light-emitting element 7 includes a light-emitting element substrate SULED, an n-type cladding layer NC, a light-emitting layer EM, a p-type cladding layer PC, an anode terminal ELED1, and the cathode terminal ELED2. The n-type cladding layer NC, the light-emitting layer EM, the p-type cladding layer PC, and the cathode terminal ELED2 are stacked on the light-emitting element substrate SULED in the order as listed. The anode terminal ELED1 is provided between the light-emitting element substrate SULED and the coupling layer CL.

The light-emitting layer EM is of, for example, indium gallium nitride (InGaN). The p-type cladding layer PC and the n-type cladding layer NC are of, for example, gallium nitride (GaN). The light-emitting element substrate SULED is of silicon carbide (SiC). Both the anode terminal ELED1 and the cathode terminal ELED2 are of aluminum.

In a manufacturing process of the light-emitting element 7, manufacturing equipment forms films of the n-type cladding layer NC, the light-emitting layer EM, the p-type cladding layer PC, and the cathode terminal ELED2 upon the light-emitting element substrate SULED. Then, the manufacturing equipment forms the light-emitting element substrate SULED into a thin film and forms the anode terminal ELED1 on the bottom surface of the light-emitting element substrate SULED. The manufacturing equipment then cuts the light-emitting element 7 into a square and disposes it upon the coupling layer CL.

With such a configuration, the anode (anode terminal ELED1) of the light-emitting element 7 is coupled to the anode power supply line IPL through the drive transistor DRT. The anode power supply line IPL is supplied with an anode power supply potential PVDD. The cathode (cathode terminal ELED2) of the light-emitting element 7 is supplied with a cathode reference potential. The anode power supply potential PVDD is a higher potential than the cathode reference potential. As a result, the light-emitting element 7 is supplied with a forward current (drive current) by a potential difference between the anode power supply potential PVDD and the cathode reference potential, and thereby, emits light. The configuration of the light-emitting element 7 illustrated in FIGS. 8 and 9 is merely an example. The light-emitting element having another configuration may be employed.

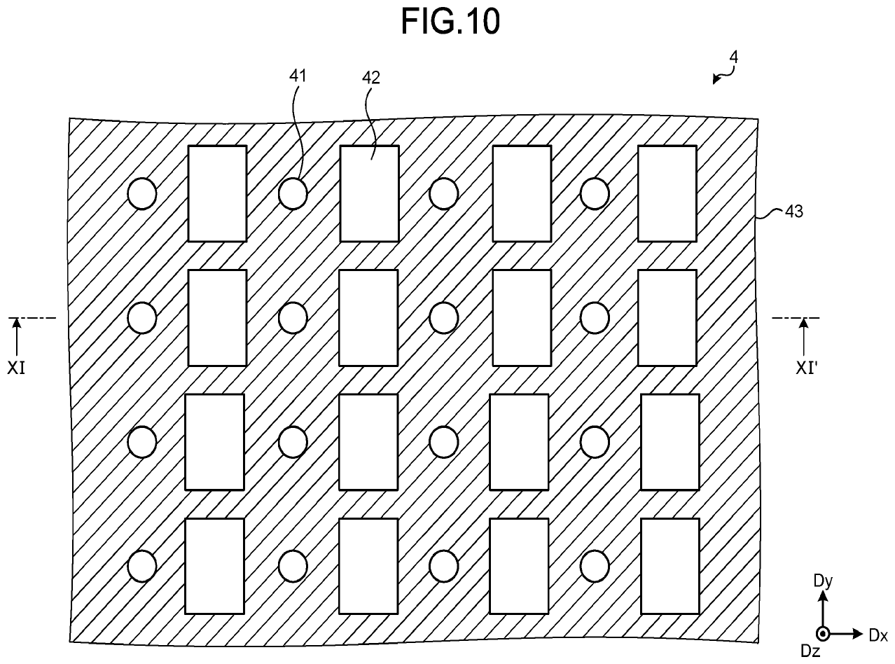

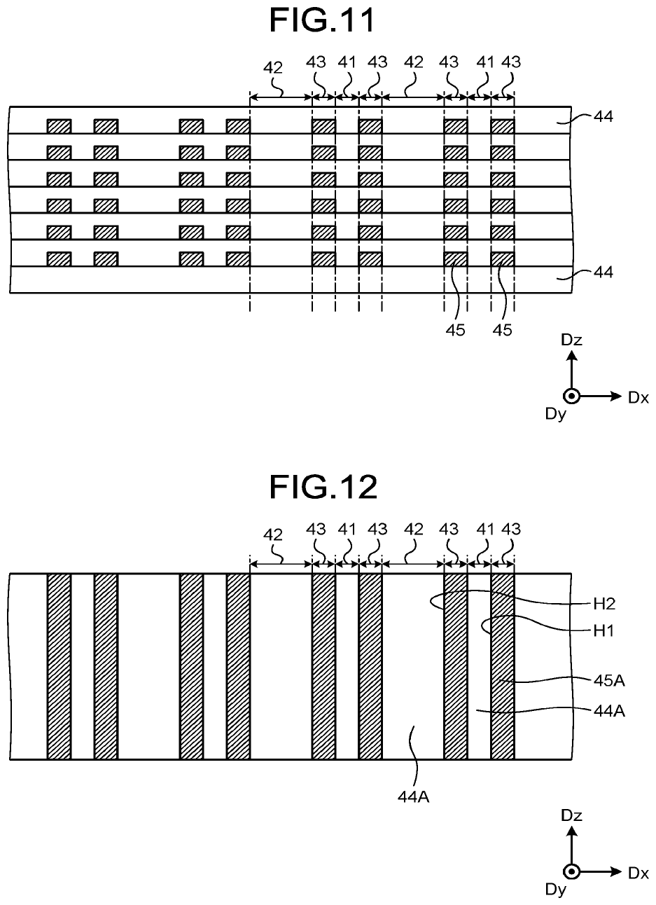

FIG. 10 is a plan view illustrating the optical element. FIG. 11 is a XI-XI′ sectional view of FIG. 10. As illustrated in FIG. 10, the optical element 4 includes the first light-transmitting areas 41, the second light-transmitting areas 42, and the non-light-transmitting area 43. The first light-transmitting areas 41 and the second light-transmitting areas 42 are provided so as to correspond to the photoelectric conversion elements 6 and the light-emitting elements 7. The first light-transmitting areas 41 and the second light-transmitting areas 42 are arranged in the first direction Dx and the second direction Dy in the plan view. Specifically, the first light-transmitting areas 41 are adjacent to the second light-transmitting areas 42 in the first direction Dx with the non-light-transmitting area 43 interposed therebetween. The first light-transmitting areas 41 and the second light-transmitting areas 42 are each arranged in the second direction Dy.

The first light-transmitting area 41 is circular in the plan view. The second light-transmitting area 42 is rectangular in the plan view. The area of the second light-transmitting area 42 is larger than the area of the first light-transmitting area 41. This configuration can restrain the extraction efficiency of the light L1 from the light-emitting elements 7 from decreasing. However, the shapes in the plan view of the first light-transmitting area 41 and the second light-transmitting area 42 may be modified as appropriate in accordance with the shape of a light-receiving surface of the photoelectric conversion element 6 and the shape of the light-emitting surface of the light-emitting element 7. The shapes of first light-transmitting area 41 and the second light-transmitting area 42 are not limited to being circular and quadrilateral, respectively, and may be, for example, polygonal, elliptical, or irregular-shaped.

As illustrated in FIG. 11, the optical element 4 includes first light-transmitting resins 44 and non-light-transmitting resins 45. The first light-transmitting resins 44 are stacked in the third direction Dz. The non-light-transmitting resins 45 are provided between layers of the first light-transmitting resins 44 in areas overlapping the non-light-transmitting area 43. Each of the first light-transmitting resins 44 is a light-transmitting resin material that transmits the visible light and the near-infrared light. Each of the non-light-transmitting resins 45 is a material having lower light transmittance than that of the first light-transmitting resin 44. The non-light-transmitting resin 45 is a colored resin material such as a black resin material.

In other words, the first light-transmitting areas 41 and the second light-transmitting areas 42 are areas not overlapping the non-light-transmitting resins 45 and are formed of only the first light-transmitting resins 44 from one surface to the other surface of the optical element 4. The non-light-transmitting area 43 is an area including at least one non-light-transmitting resin 45 between the one surface and the other surface of the optical element 4. Such a configuration allows the optical element 4 to transmit the light L1 through the first light-transmitting areas 41, transmit the light L2 through the second light-transmitting areas 42, and prevent the light L1 and L2 from transmitting through the non-light-transmitting area 43.

FIG. 12 is a sectional view illustrating the optical element according to a first modification. As illustrated in FIG. 12, in the optical element 4 according to the first modification, a non-light-transmitting resin 45A is formed into a flat plate shape and is provided with through-holes H1 and H2 in areas overlapping the first light-transmitting areas 41 and the second light-transmitting areas 42. Each of the through-holes H1 and H2 penetrates from the one surface to the other surface of the optical element 4. A first light-transmitting resin 44A is provided in each of the through-holes H1 and H2 and is formed into a column shape extending in the third direction Dz.

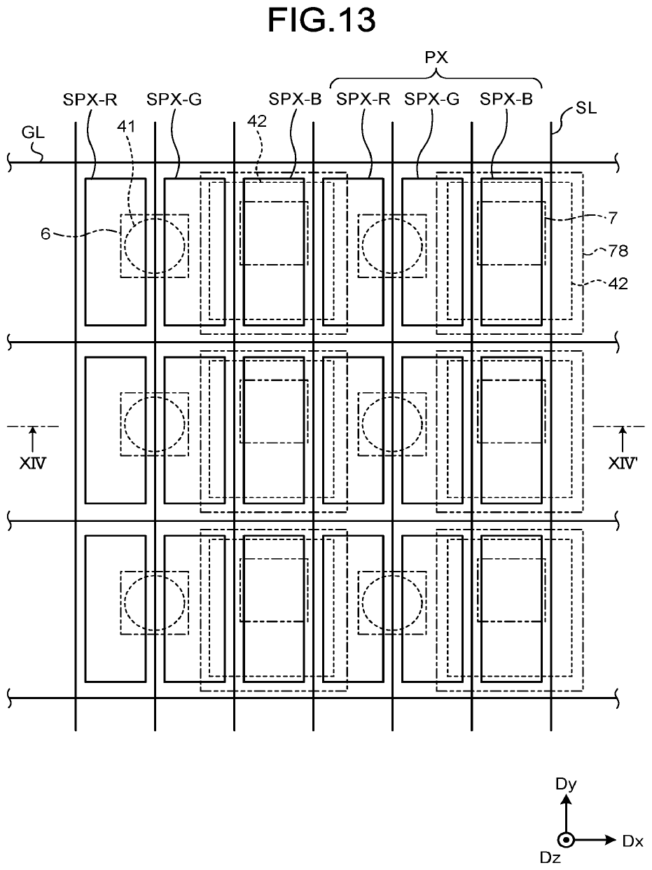

FIG. 13 is an explanatory diagram for explaining an arrangement relation between the display panel, the optical element, and the optical sensor in the plan view. In FIG. 13, dotted lines indicate the first light-transmitting areas 41 and the second light-transmitting areas 42 of the optical element 4, and long dashed double-short dashed lines indicate the photoelectric conversion elements 6, the light-emitting elements 7, and the anode electrodes 78 of the optical sensor 5.

As illustrated in FIG. 13, the photoelectric conversion element 6 and the light-emitting element 7 are arranged for each of the pixels PX. That is, one photoelectric conversion element 6 and one light-emitting element 7 are provided for each set of the sub-pixels SPX-R, SPX-G, and SPX-B. The arrangement pitch in the first direction Dx of the photoelectric conversion elements 6 is equal to the arrangement pitch in the first direction Dx of the pixels PX. The arrangement pitch in the second direction Dy of the photoelectric conversion elements 6 is equal to the arrangement pitch in the second direction Dy of the pixels PX. In the same manner, the arrangement pitch in the first direction Dx of the light-emitting elements 7 is equal to the arrangement pitch in the first direction Dx of the pixels PX, and the arrangement pitch in the second direction Dy of the light-emitting elements 7 is equal to the arrangement pitch in the second direction Dy of the pixels PX.

However, one photoelectric conversion element 6 and one light-emitting element 7 may be arranged for each set including more than one pixel PX. The arrangement pitch in the first direction Dx of the photoelectric conversion elements 6 may be an integer multiple of the arrangement pitch in the first direction Dx of the pixels PX. The arrangement pitch in the second direction Dy of the photoelectric conversion elements 6 may be an integer multiple of the arrangement pitch in the second direction Dy of the pixels PX. In the same manner, the arrangement pitch in the first direction Dx of the light-emitting elements 7 may be an integer multiple of the arrangement pitch in the first direction Dx of the pixels PX, and the arrangement pitch in the second direction Dy of the light-emitting elements 7 may be an integer multiple of the arrangement pitch in the second direction Dy of the pixels PX. In FIG. 13, one light-emitting element 7 is provided for one photoelectric conversion element 6. However, the present embodiment is not limited thereto. For example, one light-emitting element 7 may be provided for several tens to several hundreds of the photoelectric conversion elements 6 (pixels PX).

The photoelectric conversion element 6 is provided in an area overlapping at least one of the sub-pixel SPX-R for displaying the red color and the sub-pixel SPX-G for displaying the green color. In FIG. 13, the photoelectric conversion element 6 is disposed so as to overlap the sub-pixel SPX-R for displaying the red color and the sub-pixel SPX-G for displaying the green color. The luminance per unit area of the sub-pixel SPX-B for displaying the blue color is lower than that of each of the sub-pixels SPX-R and SPX-G. In the present embodiment, the light-emitting element 7 is disposed in an area overlapping the sub-pixel SPX-B. This configuration can restrain the luminance of the sub-pixel SPX-B from decreasing, and thus, can improve display characteristics of the display panel 2.

The first light-transmitting areas 41 of the optical element 4 are arranged so as to overlap the photoelectric conversion elements 6. In the plan view, the area of each of the first light-transmitting areas 41 is smaller than the area of each of the photoelectric conversion elements 6. That is, the diameter of the first light-transmitting area 41 is less than the widths WA1 and WA2 of the photoelectric conversion element 6 (refer to FIG. 5). The area of the photoelectric conversion element 6 is specifically the area of the upper electrode 65 that receives the light L2. The above-described configuration can reduce crosstalk between the photoelectric conversion elements 6, and thus, can improve detection accuracy of the optical sensor 5.

The second light-transmitting areas 42 are arranged so as to overlap the light-emitting elements 7 and the anode electrodes 78. The width in the first direction Dx and the width in the second direction Dy of the second light-transmitting area 42 are respectively less than the widths WB1 and WB2 of the anode electrode 78 (refer to FIG. 5).

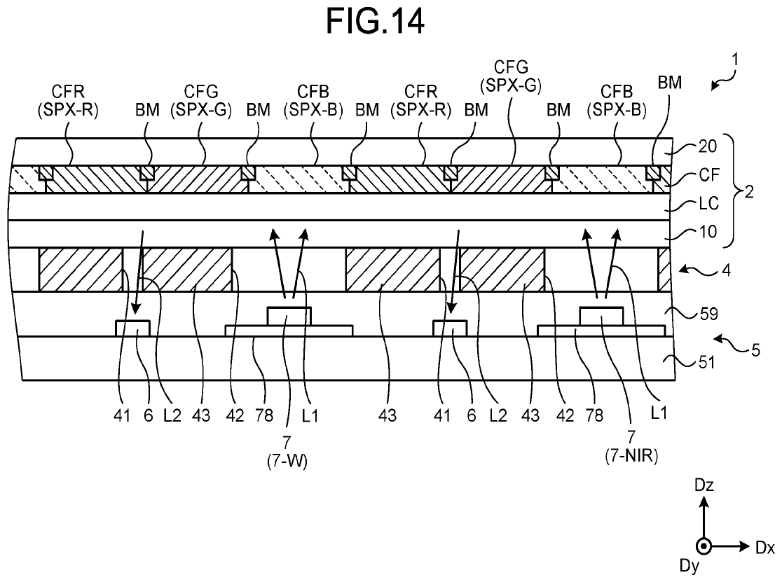

FIG. 14 is a XIV-XIV′ sectional view of FIG. 13. FIG. 14 schematically illustrates the arrangement relation between the display panel 2, the optical element 4, and the optical sensor 5 in the sectional view. As illustrated in FIG. 14, the sensor base member 51, the light-emitting element 7, the second light-transmitting area 42, the first substrate 10, the liquid crystal layer LC, the color filter CFB, and the second substrate 20 are stacked in the third direction Dz in an area provided with the light-emitting element 7, in the order as listed. The sensor base member 51, the photoelectric conversion element 6, the first light-transmitting area 41, the first substrate 10, the liquid crystal layer LC, the color filters CFR and CFG, and the second substrate 20 are stacked in the third direction Dz in an area provided with the photoelectric conversion element 6, in the order as listed.

The light L1 emitted from the light-emitting element 7 passes through the second light-transmitting area 42, the first substrate 10, the liquid crystal layer LC, the color filter CFB, and the second substrate 20, and is incident on the finger Fg. The second light-transmitting area 42 of the optical element 4 desirably has a scattering structure. In this case, the light L1 is scattered in the second light-transmitting area 42 and is emitted over the sub-pixels SPX-R and SPX-G, which are adjacent to the sub-pixel SPX-B, and more than one pixel PX. This configuration can reduce differences in luminance of the light L1 emitted from the display surface of the display panel 2, and thus, can improve the display characteristics.

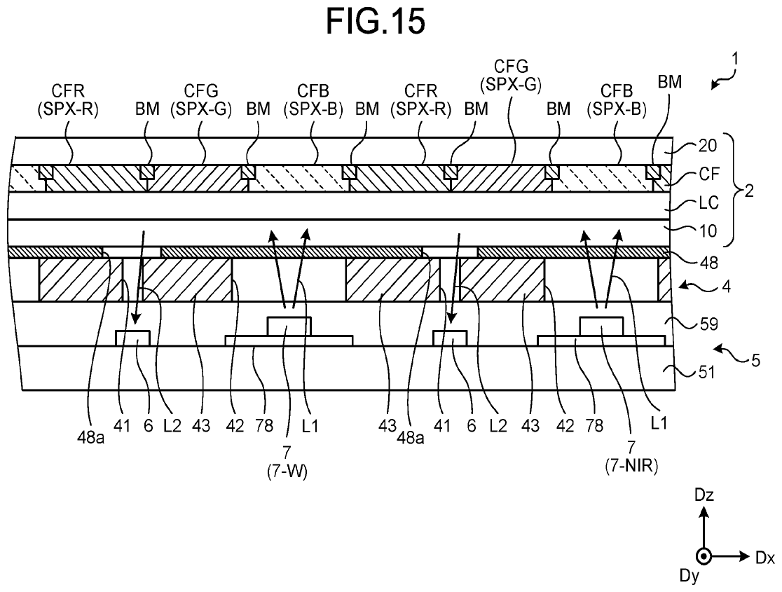

FIG. 15 is a sectional view illustrating an example of the scattering structure. As illustrated in FIG. 15, a scattering layer 48 is provided upon the optical element 4. The scattering layer 48 is provided on the upper side of the light-emitting elements 7 so as to cover at least the second light-transmitting areas 42. The scattering layer 48 scatters the light L1 from the light-emitting elements 7. The scattering layer 48 is provided with openings 48a in areas overlapping the first light-transmitting areas 41 and the photoelectric conversion elements 6. That is, the scattering layer 48 is not provided upon the first light-transmitting areas 41 and the photoelectric conversion elements 6. The scattering layer 48 is, for example, applied to be formed upon the optical element 4. The openings 48a are formed by etching. However, the method for forming the scattering layer 48 is not limited to the above-described method.

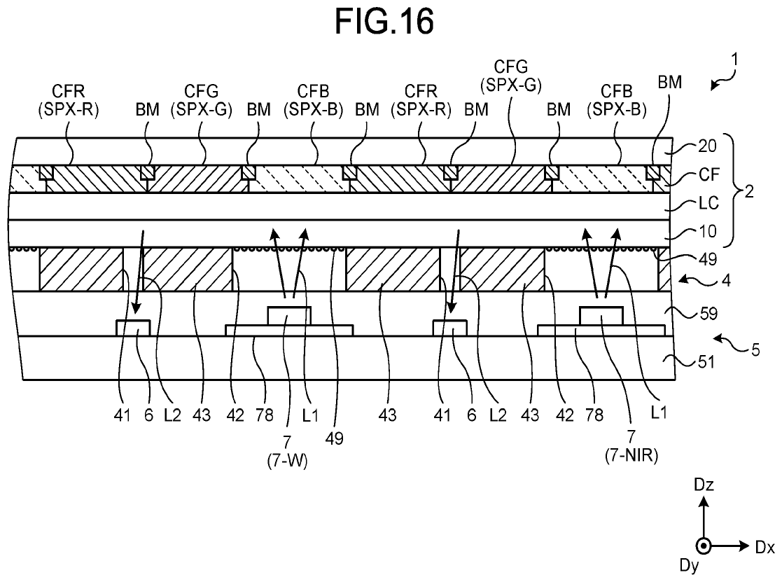

FIG. 16 is a sectional view illustrating another example of the scattering structure. As illustrated in FIG. 16, a fine asperity structure 49 is formed on a surface of the second light-transmitting area 42. A surface of the first light-transmitting area 41 is a flat surface on which the asperity structure 49 is not formed. The asperity structure 49 scatters the light L1 from the light-emitting element 7. The asperity structure 49 can be formed by covering the first light-transmitting area 41 with a metal mask, and roughening the surface of the second light-transmitting area 42 not covered with the metal mask using, for example, sandblasting or dry ice blasting. The scattering structures illustrated in FIGS. 15 and 16 can be employed in the optical element 4 of FIGS. 11 and 12.

The light L2 reflected by the finger Fg passes through the second substrate 20, the color filters CFR and CFG, the liquid crystal layer LC, the first substrate 10, and the first light-transmitting areas 41, and is incident on the photoelectric conversion elements 6. As a result, the optical sensor 5 can detect the various types of biological information such as the fingerprint and the vein pattern.

As described above, the detection device 1 of the present embodiment includes the optical sensor 5, the display panel 2 (liquid crystal display panel), the light-emitting elements 7, and the optical element 4. The optical sensor 5 includes the sensor base member 51 and the photoelectric conversion elements 6 that are provided on the sensor base member 51 and output the signals corresponding to the light emitted to the photoelectric conversion elements. The display panel 2 is provided so as to face the sensor base member 51 in the direction orthogonal to the sensor base member 51. The light-emitting elements 7 are located between the display panel 2 and the sensor base member 51 in the direction orthogonal to the sensor base member 51, and emit the light L1 (output light) to the display panel 2. The optical element 4 includes the first light-transmitting areas 41 and the non-light-transmitting area 43 and is provided between the optical sensor 5 and the display panel 2 in the direction orthogonal to the sensor base member 51. In the optical element 4, the first light-transmitting areas 41 are provided at the positions overlapping the respective photoelectric conversion elements 6 so as to penetrate the optical element 4 in the thickness direction thereof, and transmit the incident light incident on the photoelectric conversion elements 6. The non-light-transmitting area 43 is provided between the first light-transmitting areas 41 and have the light transmittance lower than that of the first light-transmitting areas 41. The light-emitting elements 7 are provided on the sensor base member 51.

With this configuration, the photoelectric conversion elements 6 and the light-emitting elements 7 are provided on the same sensor base member 51. As a result, the detection device 1 can be slimmed as compared with a configuration of providing the light source of the optical sensor 5 on a substrate different from the sensor base member 51. The light-emitting elements 7 serve as both the light source of the optical sensor 5 and the light source of the display panel 2. As a result, a backlight of the display panel 2 can be eliminated.

### Second Embodiment

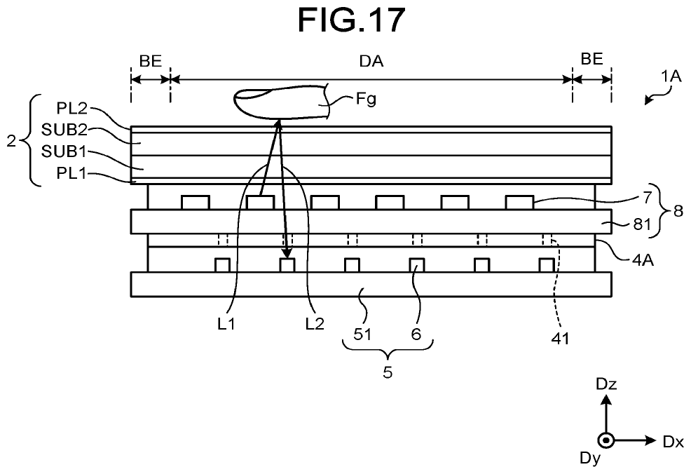

FIG. 17 is a sectional view illustrating a schematic sectional configuration of a detection device according to a second embodiment. In the following description, the components described in the above-described embodiment will be denoted by the same reference numerals, and description will be omitted.

As illustrated in FIG. 17, a detection device 1A includes the display panel 2, a lighting device 8, an optical element 4A, and the optical sensor 5. The lighting device 8 includes a light source base member 81 and the light-emitting elements 7. The light-emitting elements 7 are provided on a surface of the light source base member 81 facing the display panel 2. That is, the optical sensor 5 does not include the light-emitting elements 7, and the photoelectric conversion elements 6 are provided upon the sensor base member 51.

The lighting device 8 is provided between the optical sensor 5 and the display panel 2 in the third direction Dz. In other words, the lighting device 8 is provided between the optical sensor 5 and the finger Fg in the third direction Dz. More specifically, the detection device 1A is configured such that the optical sensor 5, the optical element 4A, the lighting device 8, and the display panel 2 are stacked in the order of the optical sensor 5, the optical element 4A, the lighting device 8, and the display panel 2 in the third direction Dz.

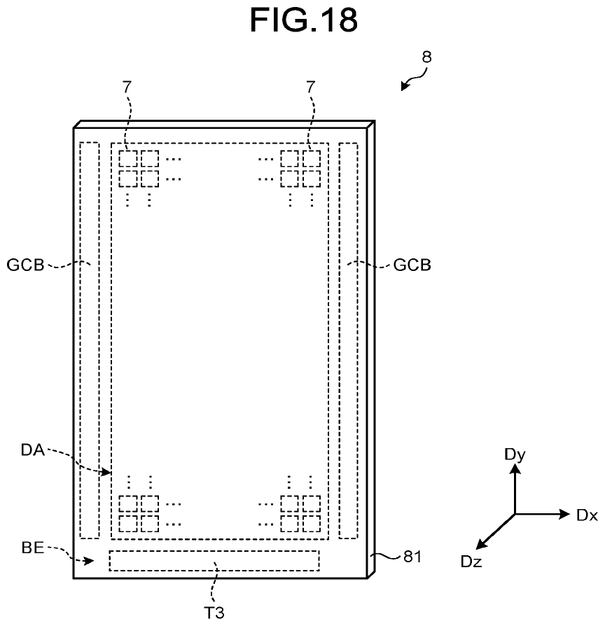

FIG. 18 is a perspective view schematically illustrating the lighting device included in the detection device according to the second embodiment. As illustrated in FIG. 18, the light-emitting elements 7 are arranged in an area of the light source base member 81 overlapping the display area DA. The light-emitting elements 7 are arranged in the first direction Dx and the second direction Dy. Peripheral circuits GCB and coupling terminals T3 for driving the light-emitting element 7 are disposed in the peripheral area BE.

The light source scan lines GLB and the light source signal lines SLB (refer to FIG. 5) are provided to the light source base member 81. The light-emitting elements 7 are provided in the areas surrounded by the light source scan lines GLB and the light source signal lines SLB. The light source scan lines GLB are coupled to the peripheral circuits GCB. The light source signal lines SLB and the peripheral circuits GCB are coupled, through the coupling terminals T3, to a control circuit and a power supply circuit for controlling the light-emitting elements 7. The arrangement relation in the plan view between the light-emitting elements 7, each of the sub-pixels SPX of the display panel 2, the first light-transmitting areas 41, and the photoelectric conversion elements 6 is the same as the configuration illustrated in FIG. 13.

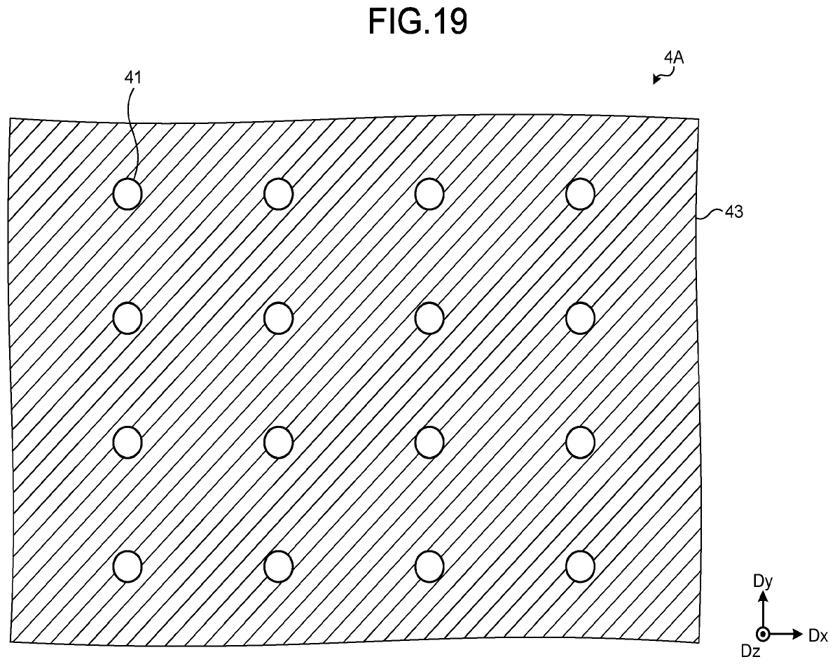

FIG. 19 is a plan view illustrating the optical element according to the second embodiment. In the present embodiment, the lighting device 8 is disposed upon the optical element 4A. Therefore, as illustrated in FIG. 19, the optical element 4A does not have the second light-transmitting areas 42. That is, areas between the first light-transmitting areas 41 adjacent in the first direction Dx serve as the non-light-transmitting area 43. The sectional configuration of the optical element 4A can employ the same configuration as that of the first embodiment illustrated in FIG. 11 or the same configuration as that of the first modification illustrated in FIG. 12. In the plan view, in the same manner as in FIG. 13, the first light-transmitting area 41 is provided in an area overlapping the photoelectric conversion element 6 and is formed so as to have a smaller area than the area of the photoelectric conversion element 6.

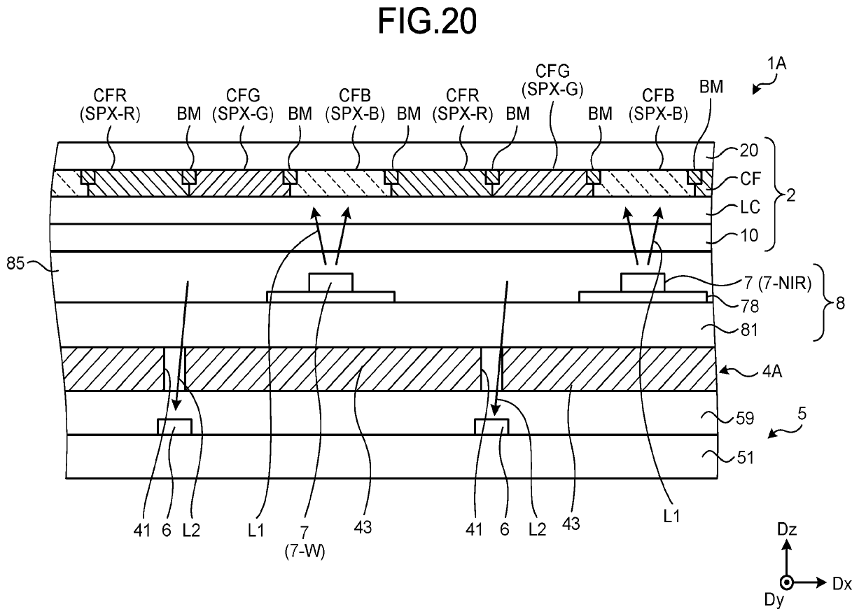

FIG. 20 is a sectional view schematically illustrating an arrangement relation between the display panel, the optical element, and the optical sensor according to the second embodiment. As illustrated in FIG. 20, the optical element 4A is provided upon the sensor base member 51 and the photoelectric conversion elements 6 of the optical sensor 5. The first light-transmitting areas 41 of the optical element 4A are provided in areas overlapping the photoelectric conversion elements 6. The non-light-transmitting area 43 of the optical element 4A is provided in an area not overlapping the photoelectric conversion elements 6.

The light source base member 81 of the lighting device 8 is provided upon the optical element 4A. The light-emitting elements 7 are provided in areas upon the light source base member 81 that overlap the non-light-transmitting area 43 of the optical element 4A. In other words, the light-emitting elements 7 are provided in areas not overlapping the first light-transmitting areas 41 and the photoelectric conversion elements 6. The display panel 2 is provided upon an overcoat layer 85 covering the light-emitting elements 7.

With the above-described configuration, the light L1 emitted from the light-emitting element 7 of the lighting device 8 passes through the first substrate 10, the liquid crystal layer LC, the color filter CFB, and the second substrate 20, and is incident on the finger Fg. The light L2 reflected by the finger Fg passes through the second substrate 20, the color filters CFR and CFG, the liquid crystal layer LC, the first substrate 10, the lighting device 8, and the first light-transmitting areas 41, and is incident on the photoelectric conversion elements 6.

In the present embodiment, the light-emitting elements 7 are provided between the optical element 4A and the display panel 2. With this configuration, the light L1 from the light-emitting elements 7 is incident on the display panel 2 without passing through the optical element 4A. Therefore, the use efficiency of the light of the light-emitting elements 7 can be improved. The photoelectric conversion elements 6 are provided in a layer different from that of the light-emitting elements 7 with the optical element 4A interposed therebetween. With this configuration, the optical element 4A can restrain the light L1 emitted to the lateral sides of the light-emitting elements 7 from being incident on the photoelectric conversion elements 6. This can improve the detection accuracy of the optical sensor 5.

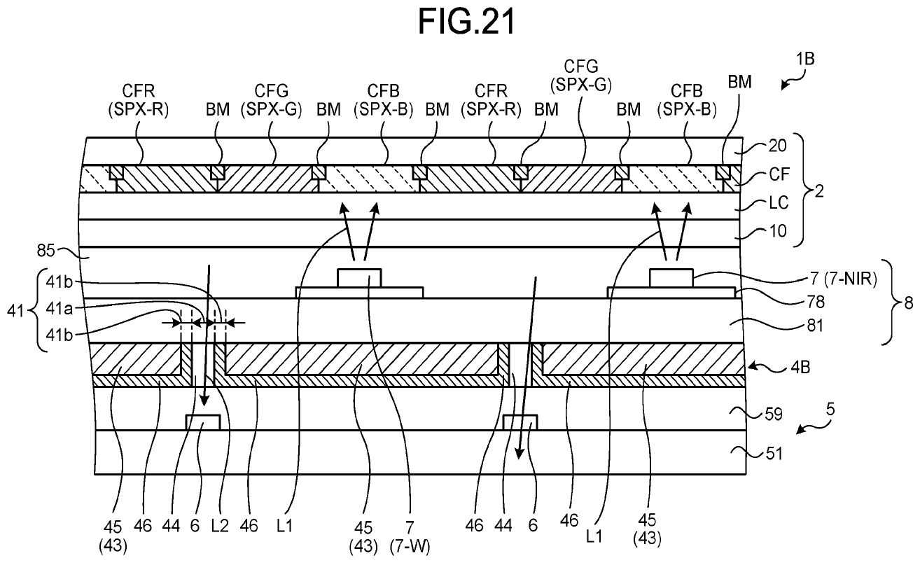

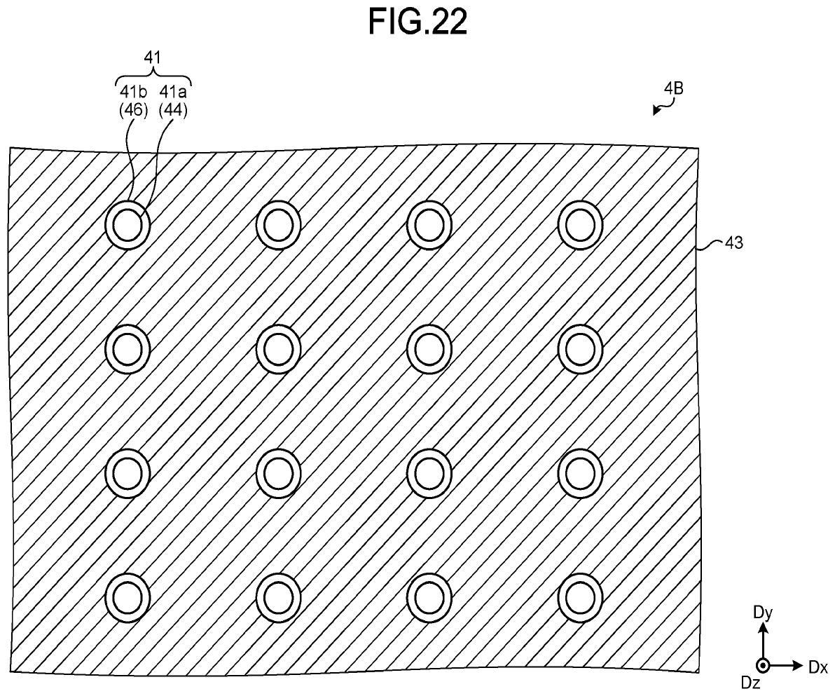

FIG. 21 is a sectional view schematically illustrating an arrangement relation between the display panel, an optical element, and the optical sensor according to a second modification of the second embodiment. FIG. 22 is a plan view illustrating the optical element according to the second modification.

As illustrated in FIG. 21, a detection device 1B of the second modification has a different configuration of an optical element 4B from that of the detection device 1A of the second embodiment. Specifically, the first light-transmitting area 41 of the optical element 4B includes a visible light transmitting area 41a and a near-infrared light transmitting area 41b. The visible light transmitting area 41a is an area that transmits the visible light and the near-infrared light. The near-infrared light transmitting area 41b is an area that does not transmit the visible light and transmits the near-infrared light.

A second light-transmitting resin 46 constituting the near-infrared light transmitting area 41b is provided so as to cover a lower surface and side surfaces of the non-light-transmitting resin 45 constituting the non-light-transmitting area 43. The first light-transmitting resin 44 constituting the visible light transmitting area 41a is provided in a through-hole provided in the second light-transmitting resin 46.

As illustrated in FIG. 22, in the plan view, the near-infrared light transmitting area 41b is formed into an annular shape surrounding the periphery of the visible light transmitting area 41a. As a result, an area obtained by combining the near-infrared light transmitting area 41b with the visible light transmitting area 41a serves as an area that can transmit the light L2 of the near-infrared light, and the visible light transmitting area 41a serves as an area that can transmit the light L2 of the visible light. That is, the area that can transmit the light L2 of the near-infrared light is larger than the area that can transmit the light L2 of the visible light. Areas between the adjacent first light-transmitting areas 41 serve as the non-light-transmitting area 43.

As described above, in performing the fingerprint detection, the first light-emitting element 7-W illustrated in FIG. 21 emits the visible light, and the light L2 reflected by the finger Fg passes through the visible light transmitting areas 41a and is incident on the photoelectric conversion elements 6. In performing the detection of the blood vessel image (vein pattern), the second light-emitting element 7-NIR emits the near-infrared light, and the light L2 reflected by the finger Fg passes through the visible light transmitting areas 41a and the near-infrared light transmitting areas 41b and is incident on the photoelectric conversion elements 6. As a result, in performing the fingerprint detection, the crosstalk can be reduced by reducing the opening diameter of the optical element 4B allowing the transmission of the light L2. In performing the detection of the blood vessel image (vein pattern) that is not required to have a resolution as high as that of the fingerprint detection, the use efficiency of the light L2 can be improved by increasing the opening diameter of the optical element 4B.

### Third Embodiment

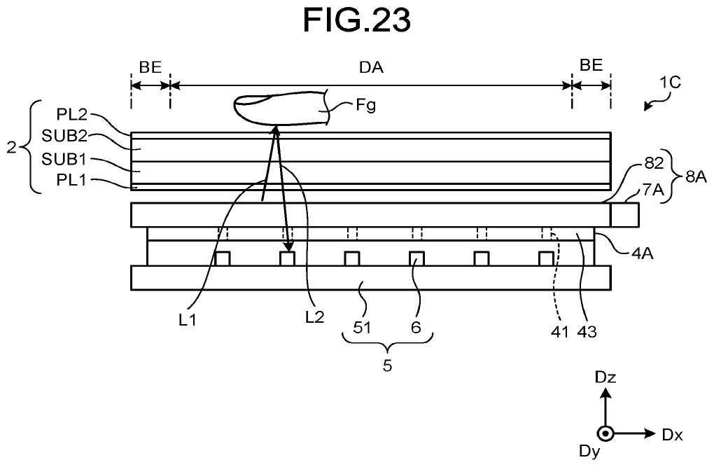

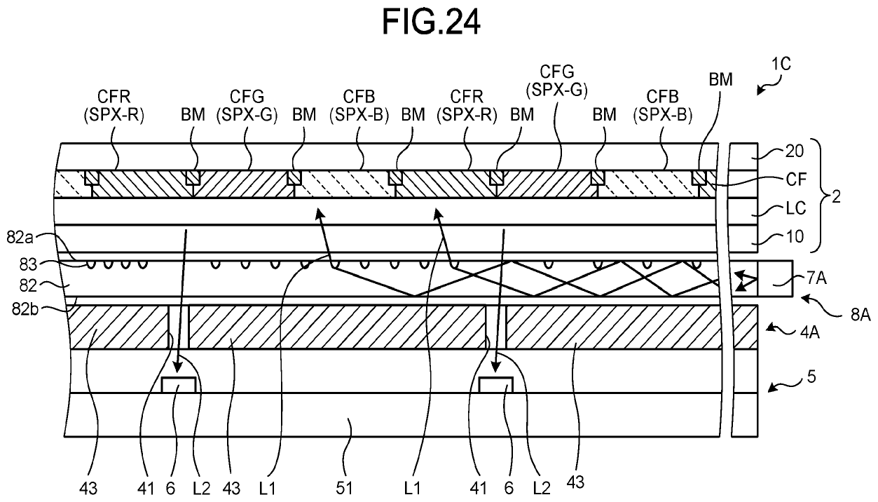

FIG. 23 is a sectional view illustrating a schematic sectional configuration of a detection device according to a third embodiment. FIG. 24 is a sectional view schematically illustrating an arrangement relation between the display panel, the optical element, and the optical sensor according to the third embodiment. As illustrated in FIG. 23, a detection device 1C of the third embodiment has a different configuration of a lighting device 8A from the first embodiment and the second embodiment described above.

The lighting device 8A includes a light guide plate 82 and a light-emitting element 7A. The light guide plate 82 has a flat plate shape and is disposed so as to face the array substrate SUB1 of the display panel 2. The light guide plate 82 is disposed in an area overlapping at least the display area DA. The light-emitting element 7A is disposed at a side end of the light guide plate 82 and emits the light L1 toward the light guide plate 82.

The stacking order of the optical sensor 5, the optical element 4A, the lighting device 8A, and the display panel 2 is the same as that in the second embodiment. Specifically, the light guide plate 82 is disposed between the optical element 4A and display panel 2 in the third direction Dz.

As illustrated in FIG. 24, the light guide plate 82 is provided so as to overlap the first light-transmitting areas 41 and the non-light-transmitting area 43 of the optical element 4A. An upper surface 82a of the light guide plate 82 is provided with a plurality of recesses 83. A scattering structure for scattering the light L1 is formed by the recesses 83. The light L1 emitted from the light-emitting element 7A travels in a direction away from the light-emitting element 7A while being repeatedly reflected in the light guide plate 82. A part of the light L1 is scattered by the recesses 83 and travels from the upper surface 82a toward the display panel 2.

The light L1 emitted from the upper surface 82a of the light guide plate 82 passes through the first substrate 10, the liquid crystal layer LC, the color filters CF, and the second substrate 20, and is incident on the finger Fg. The light L2 reflected by the finger Fg passes through the second substrate 20, the color filters CFR and CFG, the liquid crystal layer LC, the first substrate 10, the light guide plate 82 of the lighting device 8, and the first light-transmitting areas 41, and is incident on the photoelectric conversion elements 6.

In this manner, the detection device 1C is not limited to employing what is called a direct-type of the lighting device 8 and can employ the edge-light-type in which the light-emitting element 7A is provided at a side end of the light guide plate 82. In the present embodiment, as compared with the second embodiment, the light source base member 81 (refer to FIG. 17) is not provided between the optical element 4A and the display panel 2. As a result, the detection device 1C can be slimmed.

The number of the recesses 83 per unit area (arrangement density) increases with increasing distance from the light-emitting element 7A. This configuration can efficiently scatter the light L1 at positions away from the light-emitting element 7A, and thus, can restrain the light L1 from being uneven in a plane. A reflecting layer may be provided between a lower surface 82b of the light guide plate 82 and the non-light-transmitting area 43. This configuration can restrain the light L1 from being emitted outward from the lower surface 82b, and thus, can improve the use efficiency of the light L1. The light-emitting elements 7A may include the first light-emitting element 7-W and the second light-emitting element 7-NIR, and the first light-emitting element 7-W and the second light-emitting element 7-NIR may be provided at the side end of the light guide plate 82.

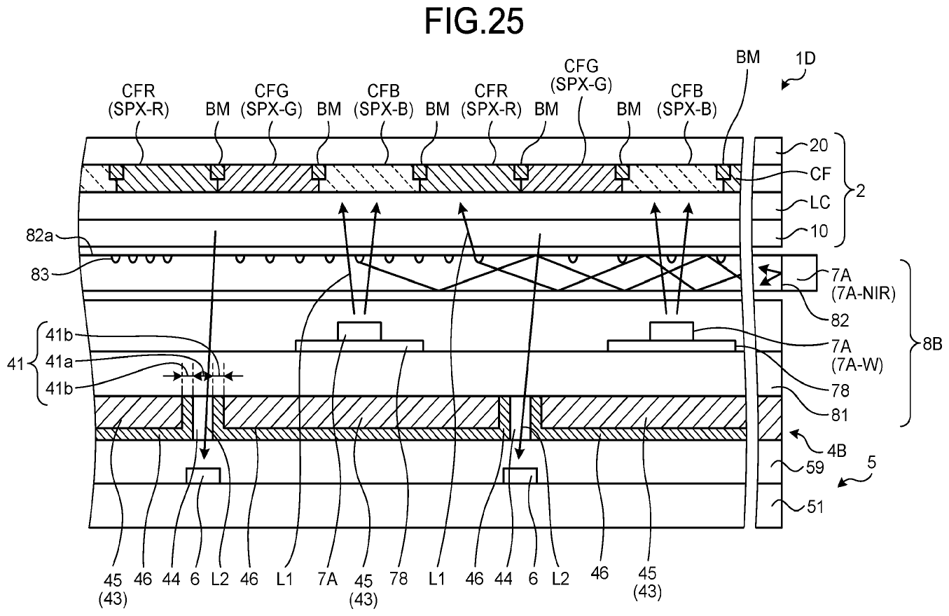

FIG. 25 is a sectional view schematically illustrating an arrangement relation between the display panel, the optical element, and the optical sensor according to a third modification of the third embodiment. In a detection device 1D according to the third modification, a lighting device 8B includes the light source base member 81, the light guide plate 82, first light-emitting elements 7A-W, and a second light-emitting element 7A-NIR. The first light-emitting elements 7A-W are provided upon the light source base member 81. The second light-emitting element 7A-NIR is provided at the side end of the light guide plate 82.

The lighting device 8B is configured such that the light source base member 81, the first light-emitting elements 7A-W, and the light guide plate 82 are stacked in the third direction Dz in the order as listed. That is, the light source base member 81 is provided upon the optical element 4B, and the light guide plate 82 is provided between the light source base member 81 and the display panel 2.

In performing the fingerprint detection, the first light-emitting elements 7A-W emit the light L1 of visible light, and the light L1 passes through the light guide plate 82 and the display panel 2 to be incident on the finger Fg. The light L2 reflected by the finger Fg passes through the display panel 2, the light guide plate 82, the light source base member 81, and the visible light transmitting areas 41a, and is incident on the photoelectric conversion elements 6. In performing the detection of the blood vessel image (vein pattern), the second light-emitting element 7A-NIR emits the near-infrared light, and the light L1 scattered by the recesses 83 of the light guide plate 82 passes through the display panel 2 to be incident on the finger Fg. The light L2 reflected by the finger Fg passes through the display panel 2, the light guide plate 82, the light source base member 81, the visible light transmitting areas 41a, and the near-infrared light transmitting areas 41b, and is incident on the photoelectric conversion elements 6.

As described above, the first light-emitting element 7A-W and the second light-emitting element 7A-NIR that emit the light L1 having different wavelengths may be provided on different members. In the third modification, the emission surface (upper surface 82a of the light guide plate 82) for emitting the light L1 of the second light-emitting element 7A-NIR is disposed at a position closer to the display panel 2 than the first light-emitting elements 7A-W are. With this configuration, the light L1 from the second light-emitting element 7A-NIR is emitted toward the display panel 2 without passing through the light source base member 81 and the first light-emitting elements 7A-W. Consequently, the detection device 1D can efficiently capture the blood vessel image (vein pattern).

While the optical element 4B includes the visible light transmitting areas 41a and the near-infrared light transmitting areas 41b in the same manner as in the second modification illustrated in FIG. 21, the configuration is not limited thereto. The detection device 1D may employ the optical element 4A of the second embodiment illustrated in FIG. 17 instead of the optical element 4B.

### Fourth Embodiment

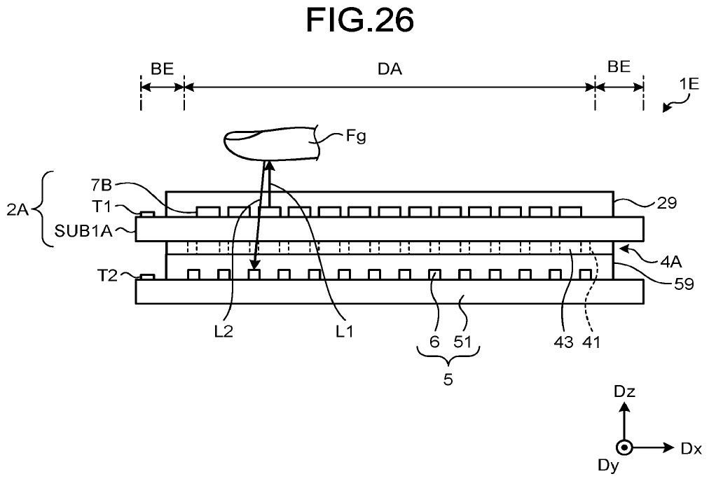

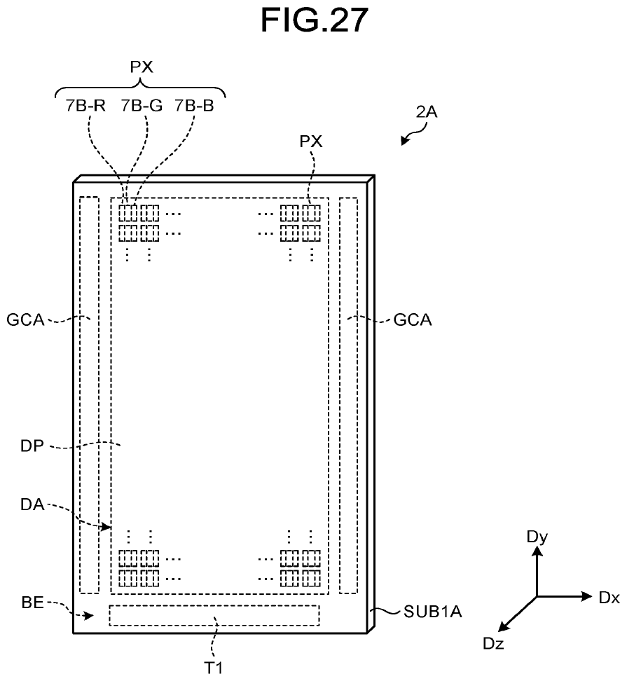

FIG. 26 is a sectional view illustrating a schematic sectional configuration of a detection device according to a fourth embodiment. FIG. 27 is a perspective view schematically illustrating a display panel included in the detection device according to the fourth embodiment.

As illustrated in FIG. 26, a detection device 1E of the fourth embodiment includes the optical sensor 5, the optical element 4A, and a display panel 2A. The optical element 4A is provided between the optical sensor 5 and the display panel 2A in the third direction Dz. The display panel 2A includes an array substrate SUB1A and a plurality of light-emitting elements 7B provided on the array substrate SUB1A. Each of the light-emitting elements 7B is an inorganic light-emitting diode (LED) chip having a size of approximately 3 μm to 100 μm in the plan view and is called a micro LED. The display panel 2A including the micro LED in each of the pixels PX is also called a micro-LED display panel. The term “micro” in the micro LED does not imply limiting the size of the light-emitting element 7B.

The sectional configuration of the array substrate SUB1A and the light-emitting elements 7B can employ the same configuration as that of FIGS. 8 and 9 illustrated in the first embodiment.

The display panel 2A includes an overcoat layer 29 covering the light-emitting elements 7B. The finger Fg comes in contact with or proximity to a surface of the overcoat layer 29. However, the present disclosure is not limited thereto. A cover substrate may be provided upon the overcoat layer 29.

As illustrated in FIG. 27, in the display panel 2A, the display area DA of the array substrate SUB1A is provided with the pixels PX. The pixels PX are arranged in the first direction Dx and the second direction Dy. Each of the pixels PX includes light-emitting elements 7B-R, 7B-G, and 7B-B. The light-emitting elements 7B-R, 7B-G, and 7B-B are arranged in the first direction Dx. The display panel 2A displays an image by emitting different light from the light-emitting elements 7B-R, 7B-G, and 7B-B. For example, the light-emitting element 7B-R emits red light; the light-emitting element 7B-G emits green light; and the light-emitting element 7B-B emits blue light.

In the following description, the light-emitting elements 7B-R, 7B-G, and 7B-B will each be simply referred to as the light-emitting element 7B when they need not be distinguished from one another. The light-emitting elements 7B may emit light in four or more different colors. The arrangement of the pixels PX and the light-emitting element 7B is not limited to the configuration illustrated in FIG. 27. For example, of the light-emitting elements 7B-R, 7B-G, and 7B-B constituting the pixel PX, two light-emitting elements 7B may be adjacent to each other in the second direction Dy.

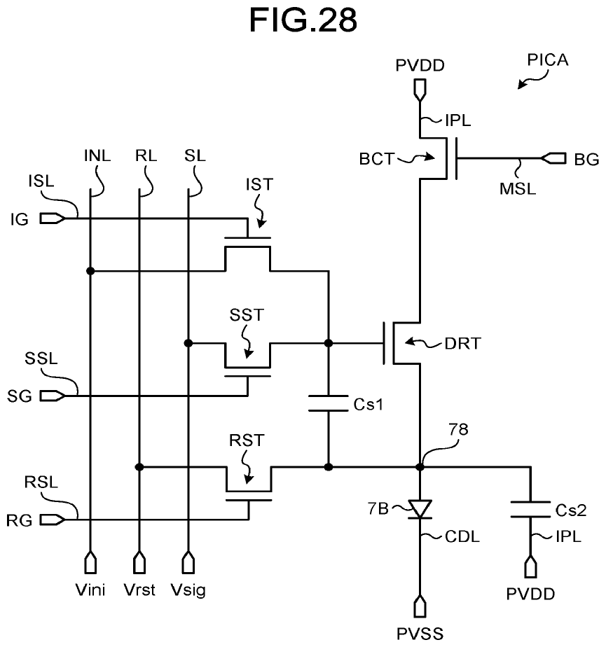

FIG. 28 is a circuit diagram illustrating a drive circuit for the light-emitting element. FIG. 28 illustrates a drive circuit PICA provided for one of the light-emitting elements 7B. The drive circuit PICA is provided for each of the light-emitting elements 7B. As illustrated in FIG. 28, the drive circuit PICA includes five transistors, and two capacitors. Specifically, the drive circuit PICA includes the drive transistor DRT, an output transistor BCT, an initialization transistor IST, a pixel selection transistor SST, and a reset transistor RST. Each of the drive transistor DRT, the output transistor BCT, the initialization transistor IST, the pixel selection transistor SST, and the reset transistor RST is formed of an n-type TFT. The drive circuit PICA also includes a first capacitor Cs1 and a second capacitor Cs2.

The cathode (cathode terminal ELED2 (refer to FIG. 9)) of the light-emitting element 7B is coupled to a cathode power supply line CDL. The anode (anode terminal ELED1 (refer to FIG. 9)) of the light-emitting element 7B is coupled to the anode power supply line IPL through the drive transistor DRT and the output transistor BCT. The anode power supply line IPL is supplied with the anode power supply potential PVDD. The cathode power supply line CDL is supplied with a cathode power supply potential PVSS. The anode power supply potential PVDD is a higher potential than the cathode power supply potential PVSS.

The anode power supply line IPL supplies the anode power supply potential PVDD serving as a drive potential to the light-emitting element 7B. Specifically, the light-emitting element 7B is supplied with a forward current (drive current) by a potential difference between the anode power supply potential PVDD and the cathode power supply potential PVSS (PVDD-PVSS), and thereby emits light. That is, the anode power supply potential PVDD has the potential difference with respect to the cathode power supply potential PVSS for causing the light-emitting element 7B to emit light. The anode terminal ELED1 of the light-emitting element 7B is coupled to the anode electrode 78, and the second capacitor Cs2 is coupled as an equivalent circuit between the anode electrode 78 and the anode power supply line IPL.

The source electrode of the drive transistor DRT is coupled to the anode terminal ELED1 of the light-emitting element 7B through the anode electrode 78, and the drain electrode of the drive transistor DRT is coupled to the source electrode of the output transistor BCT. The gate electrode of the drive transistor DRT is coupled to the first capacitor Cs1, the drain electrode of the pixel selection transistor SST, and the drain electrode of the initialization transistor IST.

The gate electrode of the output transistor BCT is coupled to an output control signal line MSL. The output control signal line MSL is supplied with an output control signal BG. The drain electrode of the output transistor BCT is coupled to the anode power supply line IPL.

The source electrode of the initialization transistor IST is coupled to an initialization power supply line INL. The initialization power supply line INL is supplied with an initialization potential Vini. The gate electrode of the initialization transistor IST is coupled to an initialization control signal line ISL. The initialization control signal line ISL is supplied with an initialization control signal IG. That is, the initialization power supply line INL is coupled to the gate electrode of the drive transistor DRT through the initialization transistor IST.

The source electrode of the pixel selection transistor SST is coupled to a video signal line SL. The video signal line SL is supplied with a video signal Vsig. A pixel control signal line SSL is coupled to the gate electrode of the pixel selection transistor SST. The pixel control signal line SSL is supplied with a pixel control signal SG.

The source electrode of the reset transistor RST is coupled to a reset power supply line RL. The reset power supply line RL is supplied with a reset power supply potential Vrst. A reset control signal line RSL is coupled to the gate electrode of the reset transistor RST. The reset control signal line RSL is supplied with a reset control signal RG. The drain electrode of the reset transistor RST is coupled to the anode terminal ELED1 of the light-emitting element 7B and the source electrode of the drive transistor DRT.

The first capacitor Cs1 is provided as an equivalent circuit between the drain electrode of the reset transistor RST and the gate electrode of the drive transistor DRT. The drive circuit PICA can reduce a variation in gate voltage of a parasitic capacitance and a leakage current of the drive transistor DRT by the first capacitor Cs1 and the second capacitor Cs2.

The gate electrode of the drive transistor DRT is supplied with a potential corresponding to the video signal Vsig (or a gradation signal). That is, the drive transistor DRT supplies a current corresponding to the video signal Vsig to the light-emitting element 7B based on the anode power supply potential PVDD supplied through the output transistor BCT. In this manner, the anode power supply potential PVDD supplied to the anode power supply line IPL is lowered by the drive transistor DRT and the output transistor BCT. As a result, the anode terminal ELED1 of the light-emitting element 7B is supplied with a potential lower than the anode power supply potential PVDD.

One electrode of the second capacitor Cs2 is supplied with the anode power supply potential PVDD through the anode power supply line IPL, and the other electrode of the second capacitor Cs2 is supplied with the potential lower than the anode power supply potential PVDD. That is, the one electrode of the second capacitor Cs2 is supplied with the potential higher than that of the other electrode of the second capacitor Cs2. For example, the one electrode of the second capacitor Cs2 is the anode power supply line IPL, and the other electrode of the second capacitor Cs2 is the anode electrode 78 and an anode coupling electrode coupled thereto.

In the display panel 2A, peripheral circuits GCA (refer to FIG. 27) sequentially select pixel rows from the top row (for example, a pixel row located at the top in the display area DA in FIG. 27) down. The drive IC writes the video signal Vsig (video writing potential) to each of the pixels PX in the selected pixel row to cause the light-emitting element 7B to emit the light. For each horizontal scan period, the drive IC supplies the video signals Vsig to the video signal lines SL, supplies the reset power supply potential Vrst to the reset power supply lines RL, and supplies the initialization potential Vini to the initialization power supply lines INL. In the display panel 2A, these operations are repeated for each frame image.

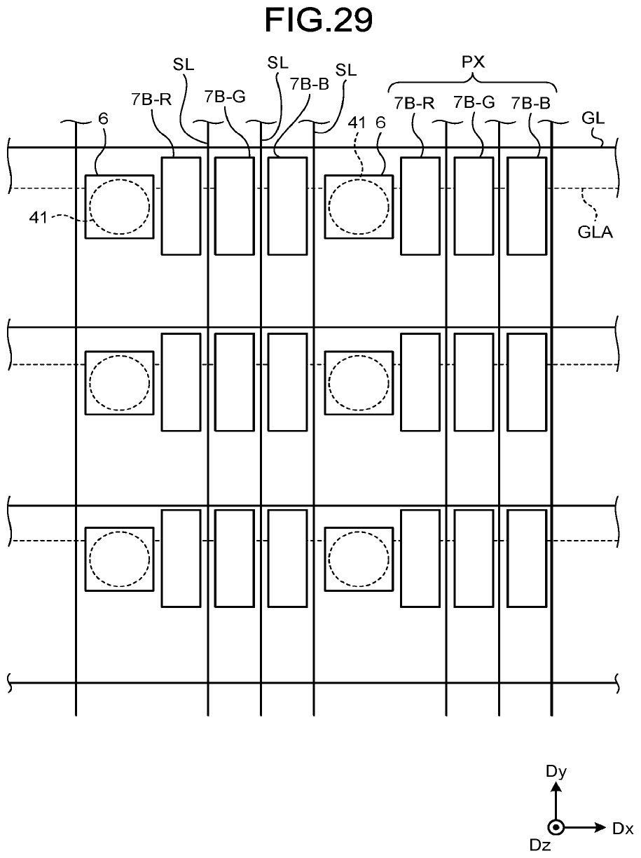

FIG. 29 is an explanatory diagram for explaining an arrangement relation in the plan view between the display panel, the optical elements, and the optical sensor according to the fourth embodiment. As illustrated in FIG. 29, the light-emitting elements 7B are provided in positions not overlapping the photoelectric conversion elements 6 and the first light-transmitting areas 41. In other words, the light-emitting elements 7B are provided in areas overlapping the non-light-transmitting area 43 of the optical element 4A (refer to FIG. 26). More than one of the light-emitting elements 7B are arranged between two of the photoelectric conversion elements 6 adjacent to each other in the first direction Dx. The arrangement is repeated in the first direction Dx, such as the photoelectric conversion element 6 and the first light-transmitting area 41, the light-emitting element 7B-R, the light-emitting element 7B-G, the light-emitting element 7B-B, the photoelectric conversion element 6 and the first light-transmitting area 41, the light-emitting element 7B-R, the light-emitting element 7B-G, the light-emitting element 7B-B. The light-emitting elements 7B the colors of which are the same are arranged in the second direction Dy such that the light-emitting elements 7B-R are arranged in the second direction Dy, the light-emitting elements 7B-G are arranged in the second direction Dy, and the light-emitting elements 7B-B are arranged in the second direction Dy.

With the above-described configuration, the light L1 emitted from each of the light-emitting elements 7B of the display panel 2A travels toward the finger Fg, as illustrated in FIG. 26. The light L2 reflected by the finger Fg passes through openings between the light-emitting elements 7B and the first light-transmitting areas 41 and is incident on the photoelectric conversion elements 6. The openings refer to areas in areas of the display panel 2A surrounded by the pixel signal lines SL and the scan lines GL that are not covered with the light-emitting elements 7B, the anode electrodes 78, and various types of wiring.

In the detection device 1E of the fourth embodiment, the light-emitting elements 7B serving as display elements of the display panel 2A also serve as the light source of the optical sensor 5. Therefore, the detection device 1E can be made smaller (slimmer) than the case of the first to the third embodiments.

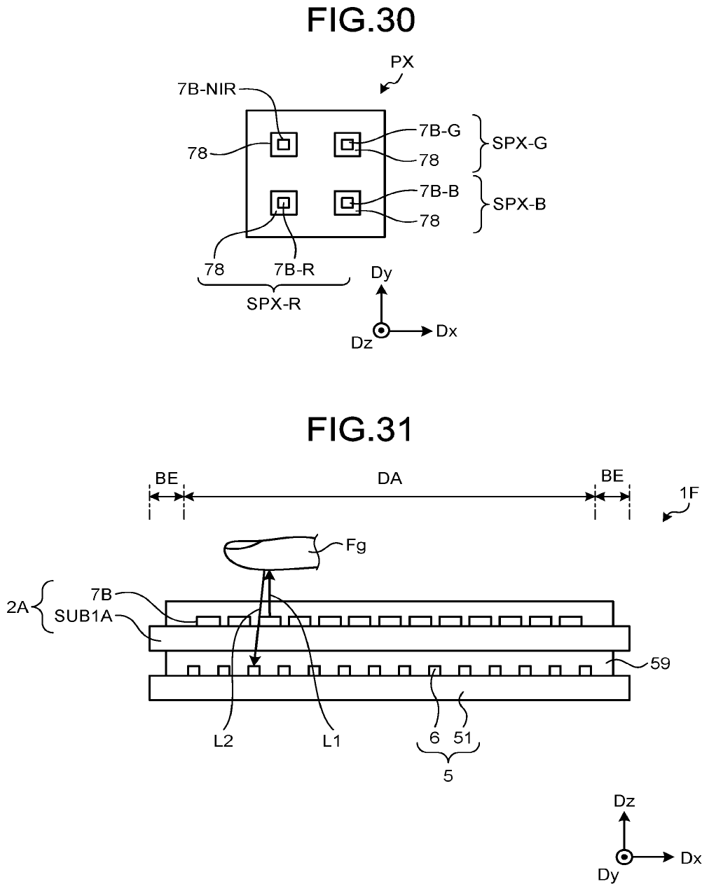

FIG. 30 is an explanatory diagram for explaining an arrangement in the pixel according to a fourth modification of the fourth embodiment. While FIG. 29 illustrates an example in which the light-emitting element 7B-R, the light-emitting element 7B-G, and the light-emitting element 7B-B constituting the pixel PX are arranged in the first direction Dx, the arrangement is not limited to this example. As illustrated in FIG. 30, in the fourth modification, the pixel PX includes a light-emitting element 7B-NIR. The light-emitting element 7B-NIR is an inorganic light-emitting element that emits infrared light, more preferably near-infrared light.

In the first direction Dx, the light-emitting element 7B-NIR is arranged adjacent to the light-emitting element 7B-G. In the second direction Dy, the light-emitting element 7B-NIR is arranged adjacent to the light-emitting element 7B-R. In the second direction Dy, the light-emitting element 7B-G is arranged adjacent to the light-emitting element 7B-B. In the first direction Dx, the light-emitting element 7B-R is arranged adjacent to the light-emitting element 7B-B.

The arrangement of the light-emitting elements 7B-NIR, 7B-R, 7B-G, and 7B-B is not limited to the example illustrated in FIG. 30. Some of the light-emitting elements 7B-NIR, 7B-R, 7B-G, and 7B-B may be replaced with one another. The light-emitting elements 7B-NIR, 7B-R, 7B-G, and 7B-B may be arranged in the first direction Dx.

The display of the display panel 2A and the detection of the optical sensor 5 may be performed in a time division manner or in a simultaneous manner. Since the light-emitting element 7B-NIR emits the invisible light, the display characteristics are not much affected even when the light-emitting element 7B-NIR emits the light L1 during a display period in which the display is performed by the light-emitting elements 7B-R, 7B-G, and 7B-B. Therefore, the optical sensor 5 can detect the biological information based on the light emitted from the light-emitting element 7B-NIR during the display period.

FIG. 31 is a sectional view illustrating a schematic sectional configuration of a detection device according to a fifth modification of the fourth embodiment. A detection device 1F according to the fifth modification has a configuration not provided with the optical element 4A, while the detection device 1E illustrated in FIG. 26 is provided therewith.

That is, as illustrated in FIG. 31, the display panel 2A including the light-emitting elements 7B (micro-LED) is provided upon the optical sensor 5 without interposing the optical element 4A therebetween. More specifically, the array substrate SUB1A is in contact with an upper surface of the overcoat layer 59 of the optical sensor 5. However, a gap may be provided between the array substrate SUB1A and the overcoat layer 59.

Also in the fifth modification, the light L1 emitted from the light-emitting element 7B travels toward the finger Fg. The light L2 reflected by the finger Fg passes through the openings of the array substrate SUB1A and is incident on the photoelectric conversion elements 6. As a result, the detection device 1F can detect the information on the living body. In the fifth modification, the optical element 4A is not provided. Therefore, the detection device 1F can be slimmed as compared with the fifth embodiment.

While the preferred embodiments of the present disclosure have been described above, the present disclosure is not limited to the embodiments described above. The content disclosed in the embodiments is merely exemplary, and can be variously modified within the scope not departing from the gist of the present disclosure. Any modification appropriately made within the scope not departing from the gist of the present disclosure also naturally belongs to the technical scope of the present disclosure.

What is claimed is:

1. A detection device comprising:
an optical sensor comprising a sensor base member and a plurality of photoelectric conversion elements that are provided on the sensor base member and configured to output signals corresponding to light emitted to the photoelectric conversion elements;
a light-emitting element configured to emit output light toward a direction of an object to be measured; and
an optical element comprising a plurality of first light-transmitting areas and a non-light-transmitting area, and provided between the optical sensor and the object to be measured, wherein
in the optical element,
the first light-transmitting areas are provided at positions overlapping the respective photoelectric conversion elements so as to penetrate the optical element in a thickness direction of the optical element and are configured to transmit incident light incident on the photoelectric conversion elements, and
the non-light-transmitting area is provided between the first light-transmitting areas and has light transmittance lower than light transmittance of the first light-transmitting areas.

2. The detection device according to claim 1, wherein an area of each of the first light-transmitting areas is smaller than an area of each of the photoelectric conversion elements.

3. The detection device according to claim 1, wherein a plurality of the light-emitting elements are provided on the sensor base member and are provided adjacent to the respective photoelectric conversion elements in a plan view.

4. The detection device according to claim 3, wherein
the optical element comprises a plurality of second light-transmitting areas adjacent to the first light-transmitting areas with the non-light-transmitting area interposed between the first light-transmitting areas and the second light-transmitting areas, and
the second light-transmitting areas are provided at positions overlapping the light-emitting elements and are configured to transmit the output light.

5. The detection device according to claim 1, comprising a lighting device comprising a plurality of the light-emitting elements and a light source base member provided with the light-emitting elements, wherein
the lighting device is provided between the optical element and the object to be measured in a direction orthogonal to the sensor base member.

6. The detection device according to claim 5, wherein the light-emitting elements are provided in areas overlapping the non-light-transmitting area of the optical element.

7. The detection device according to claim 1, comprising a liquid crystal display panel provided between the light-emitting element and the object to be measured, wherein
the liquid crystal display panel comprises a red pixel configured to display a red color, a green pixel configured to display a green color, and a blue pixel configured to display a blue color,
the light-emitting element is provided in an area overlapping the blue pixel, and
each of the photoelectric conversion elements is provided in an area overlapping at least one of the red pixel and the green pixel.

8. The detection device according to claim 1, comprising a lighting device comprising a light guide plate and the light-emitting element provided at a side end of the light guide plate, wherein
the lighting device is provided between the optical element and the object to be measured in a direction orthogonal to the sensor base member.

9. The detection device according to claim 8, wherein
the lighting device further comprises a light source base member provided between the light guide plate and the optical element, and
a plurality of the light-emitting elements comprise a plurality of first light-emitting elements provided on the light source base member and configured to emit visible light, and a second light-emitting element provided at the side end of the light guide plate and configured to emit near-infrared light.

10. The detection device according to claim 1, wherein a plurality of the light-emitting elements comprise a plurality of first light-emitting elements configured to emit visible light and a second light-emitting element configured to emit near-infrared light.

11. The detection device according to claim 1, wherein the photoelectric conversion elements are positive-intrinsic-negative (PIN) diodes.

12. The detection device according to claim 1, wherein the light-emitting element is an inorganic light-emitting element or an organic light-emitting diode (OLED).

13. A detection device comprising:
an optical sensor comprising a sensor base member and a plurality of photoelectric conversion elements that are provided on the sensor base member and configured to output signals corresponding to light emitted to the photoelectric conversion elements;
a display panel comprising a plurality of inorganic light-emitting elements arranged on an array substrate; and
an optical element comprising a plurality of first light-transmitting areas and a non-light-transmitting area, and provided between the optical sensor and the display panel in a direction orthogonal to the sensor base member, wherein
in the optical element,
the first light-transmitting areas are provided at positions overlapping the respective photoelectric conversion elements so as to penetrate the optical element in a thickness direction of the optical element and are configured to transmit incident light incident on the photoelectric conversion elements, and
the non-light-transmitting area is provided between the first light-transmitting areas and has light transmittance lower than light transmittance of the first light-transmitting areas.

14. The detection device according to claim 13, wherein
the inorganic light-emitting elements are provided in areas overlapping the non-light-transmitting area in a plan view as viewed from a direction orthogonal to the sensor base member, and
more than one of the inorganic light-emitting elements is arranged between adjacent two of the photoelectric conversion elements.
